(19) United States
(12) Patent Application Publication
(10) Pub. No.: US 2026/0363270 A1
(43) Pub. Date: Nov. 19, 2026

(54) CELL COMPOSITIONS

(71) Applicant: GIVAUDAN SA, VERNIER (CH)

(72) Inventors: Jay Patrick SLACK, Cincinnati, OH (US); Thomas Scott MCCLUSKEY, Cincinnati, OH (US); Stijn ROTMAN, Kemptthal (CH); Elisa GALLO, Kemptthal (CH); Sabine VOLLENWEIDER, Kemptthal (CH); Omer GIL, Kemptthal (CH)

(21) Appl. No.: 19/126,095

(22) PCT Filed: Nov. 8, 2023
(86) PCT No.: PCT/EP2023/081186

§ 371 Date: Apr. 30, 2025

Related U.S. Application Data
(60) Provisional application No. 63/382,935, filed on Nov. 9, 2022.

(51) Int. Cl. A23J 3/22; A23J 3/04; A23L 2/56; A23L 13/40; A23L 27/00; A23L 27/10; C12N 5/07

(52) U.S. Cl. CPC A23J 3/227; A23J 3/04; A23L 2/56; A23L 13/45; A23L 27/10; A23L 27/70; C12N 5/06

(57) ABSTRACT

Non-human animal-derived cell composition comprising a plurality of non-human animal-derived cells, wherein the cells comprise at least one exogenous supplement comprised in a carrier selected from the group consisting of nanocarriers, nanoparticles, micelles, liposomes or vesicles is described. The cell composition is characterized by having improved organoleptic properties, such as an improved flavour, colour, texture or mouthfeel and/or having a meat like flavour. Methods of producing the same and comestibles comprising the same are also described.

### TECHNICAL FIELD

The present disclosure relates to the field of cultivated meat. More particularly, the disclosure relates to methods and compositions for improving the sensory properties of cultivated meat, including its colour, smell, taste, aroma, texture or mouthfeel, such that it resembles whole-animal-derived meat.

### BACKGROUND

Many valuable resources are exploited in order to sustain livestock production, including water, grain, land and energy, and the demands of the world's rapidly growing population will lead to even greater exploitation of these valuable resources. Accordingly, the ability to produce meat and meat products in ways that will reduce our reliance upon non-human animals is a highly desirable goal. Replacing animals such as cattle, sheep, pigs and poultry as the main meat sources, as well as fish and shellfish, is also ethically beneficial since it reduces the sometimes cruel conditions attendant with intensive livestock production and farming. Cultivated meat production also provides options for consumers who abstain from eating meat on ethical grounds, and for those consumers that freely consume meat today, it can offer additional health benefits in that the production of cultivated meat does not require the use of growth hormones and antibiotics.

However, creating some or all of the sensory properties of whole meat in cultivated products, such that these products resemble whole meat, remains a challenging task.

There remains a need to provide cultivated meat products that can offer the consumer an alternative to meat consumption without compromising on any of the beneficial or sensorial properties of intensively farmed meat.

Development of food products containing cultivated meat shows that there is still a huge gap between food products from cultivated meat and meat from livestock in terms of nutritional value, both macro and micro nutritional, flavour, colour, sense and texture of the final food product. Food products from cultivated meat are nowhere near the characteristics of intensively farmed meat and its food products. WO2020/100143 (ALEPH Farms) took first steps in this direction, in particular, to increase the micro nutritional value of the final food product, but also to contribute to a more realistic meat flavour of the cultivated meat. WO 2015/038988 (Modern Meadows) discloses millimetre sized edible microcarriers, which are used as scaffolds for adherent cells to latch or adsorb onto and avoiding need to separate said microcarriers from the cell culture medium.

The goal of the present disclosure is to incorporate molecules such as flavour compounds, flavour precursors, colourants and/or nutrients in the cell cultures, to provide a cultivated raw meat product having organoleptic properties and nutritional content as similar as possible to meat from livestock.

However, intensive investigations of the applicant have shown that flavours cannot be incorporated in cell cultures directly due to excretory and breakdown processes of the cell starting immediately when exogenous, i.e. non-cellular or non-physiological, contents are brought in contact with the cell and the cell medium. Furthermore, hydrophobicity and cytotoxic nature of some compounds may also present problems for their incorporation.

Thus, there is a need to make non-physiological and exogenous compounds available directly in the cells to become immersed and encompassed or embedded in the final cultivated raw meat and subsequently the final food product to provide characteristic meat properties of raw or cooked meat, like meaty flavours, flavour precursors or colours, possibly providing the characteristic colour change upon cooking, or supplements like nutraceuticals, proteins or other bioactive compounds, or supplements providing further typical characteristics of meat such as fat, texture and mouthfeel.

To avoid excretion and degradation of the compounds, other means to deliver exogenous compounds to the cell and to keep it in the cell, are required.

Possible methods of delivery include but are not limited to transduction, electroporation, nanoparticle delivery, and liposomal delivery.

Liposomes have been used in medicinal and pharmaceutical research, particularly in targeted drug delivery to different sites in the body, such as different tissues in the body, such as connective tissue, or to cells including but not limited to endothelial cells, e.g. blood vessels, with e.g. via pH control or other mechanisms: But there has also been published research using liposomes in mitochondrial delivery of actives directly to the cell (“MITO-Porter” and “Mitochondrial Delivery system using liposomes as nanocarriers that target myoblast cells”).

In food science, liposomes have been used for different purposes like, delivery and preservation of nutrients, or bioactives, for encapsulation and packaging, e.g. in “Application of Liposomes in the Food Industry, Z. Mirafzali, C. S. Thompson, K. Tallua in Microencapsulation in the Food Industry—A Practical Implementation Guide, 2014, Chapter 13, p. 139-150”, in “Antioxidant activity of spice extracts in a liposome system and in cooked pork patties and the possible mode of action; By: Kong, Baohua; Zhang, Huiyun; Xiong, Youling L. Meat Science (2010), 85(4), 772-778”; Or for emulsifying: e.g. in “Dominguez, R.; Pateiro, M.; Munekata, P. E. S.; McClements, D. J.; Lorenzo, J. M. Encapsulation of Bioactive Phytochemicals in Plant-Based Matrices and Application as Additives in Meat and Meat Products. Molecules 2021, 26, 3984”.

Liposomes have been used successfully in delivery of proteins into cells, e.g. in “Fu et al. “Promises and Pitfalls of Intracellular Delivery of Proteins, Bioconjugate Chem. 2014, 25, 1602-1608”; or in “Nahum, V.; Domb, A. J. Recent Developments in Solid Lipid Microparticles for Food Ingredients Delivery. Foods 2021, 10, 400”; or in “Sharma, S.; Mulrey, L.; Byrne, M.; Jaiswal, A. K.; Jaiswal, S. Encapsulation of Essential Oils in Nanocarriers for Active Food Packaging. Foods 2022, 11, 2337”; or in “Akbarzadeh et al. Nanoscale Research Letters 2013, 8:102.

### SUMMARY

According to a first illustrative aspect, provided are cell compositions comprising cultured non-human animal-derived cells that are enriched with exogenous supplements providing, or capable of providing, meat-like organoleptic properties or meat-like colouring to said cell compositions, wherein the exogenous supplements are comprised in a carrier. According to certain illustrative aspects, the exogenous supplements are comprised in the form of nanocarrier, nanoparticle, micellar, liposomal or vesicle compositions.

According to a second illustrative aspect, provided is a cultivated meat containing said exogenous supplement compositions.

According to a third illustrative aspect, provided is a comestible product containing said composition of cultivated meat.

According to a fourth illustrative aspect, provided is a cell culture media enriched with exogenous supplements provided for or comprised in a carrier, such as, without limitation, a nanocarrier, nanoparticle, micellar, liposomal or vesicle composition.

According to a fifth illustrative aspect, provided is a scaffold useful in the preparation of cultivated meat that is enriched with exogenous supplements comprised in a carrier, such as, without limitation, a nanocarrier, nanoparticle, micellar, liposomal or vesicle composition providing or capable of providing meat-like organoleptic properties to said scaffold and/or to the cells cultivated in said scaffold.

The present disclosure is based in part on the surprising and unexpected discovery that culturing cells in a medium containing exogenous supplements, such as flavours, flavour precursors or flavour enhancers, colours etc., results in the uptake and accumulation and/or adherence of the flavours or precursors if they are provided for in a nanocarrier, nanoparticle, micellar, liposomal or vesicle composition in or on the cells, thereby providing taste, aroma or colour to cultivated meat that is superior to the effect that can be achieved by simply injecting or coating a cultivated meat product with such flavouring or colouring compounds, such that cultivated meat produced according to methods of the present disclosure possess organoleptic properties, such as flavour, flavour precursors, colour and nutritional content that substantially resembles that of whole-organism derived meat. Furthermore, hydrophobic (e.g. C Log P value >2) and cytotoxic exogenous supplements can be easily and efficiently made available for uptake and accumulation and/or adherence into or to the cells.

The disclosed compositions, comestibles, and methods provide advantages and improvements over the prior art by controlling the content of exogenous supplements added to or generated in culture media and/or scaffolds used in conjunction therewith by providing them in the form of a nanocarrier, nanoparticle, a micellar, liposomal or vesicle composition, as a supplement composition or more particularly an exogenous supplement composition. Creation of flavour is achieved by administering, generating or enhancing the required amount of the exogenous supplements comprised in a carrier, for example, without limitation, in such a nanocarrier, nanoparticle, micellar, liposomal or vesicle composition during growth, proliferation and/or differentiation of the cells, thereby efficiently controlling the uptake of the supplement composition into and/or onto the cells, or into and/or onto any scaffolds used therewith. Moreover, through the judicious selection of exogenous supplement compositions, or exogenous supplements per se, added to or generated in the cell culture media and/or scaffold, it is possible to impart, modify or improve the flavour of cultivated meat using non-artificial, non-synthetic, simple ingredients that consumers recognise as wholesome and thereby may meet so-called “clean label” requirements or expectations as they relate to flavour.

It is to be explicitly understood that a cell or a plurality of cells comprising an amount of one or more exogenous supplements in accordance with the present disclosure, refers to a non-human animal cell or a plurality of such cells combined with the one or more exogenous supplement compositions in accordance with the present invention either intracellularly, within the cell membrane, adhered to the cell membrane or other cell parts and any combination thereof, provided that it is provided for in a nanocarrier, nanoparticle, micellar, liposomal or vesicle composition.

The exogenous supplements described in the present disclosure are food-grade supplements that are suitable for human consumption. The exogenous supplements can be bio-sourced, that is, they can be extracted and/or derived from a natural source, such as plants, fungi, bacteria, algae or animal sources. They may be native, i.e. extracted unmodified from their natural state, or taken from their natural state and purified, or even chemically or biochemically modified, or they can be synthesized materials that are identical to materials found in nature.

Other aspects, embodiments, features, and advantages of the present disclosure will be further described in the following detailed description of the illustrative aspects and embodiments.

### DETAILED DESCRIPTION

In recent decades, the desirability of providing cultivated meat and comestible products containing it has been acknowledged, yet high quality, affordable products are currently not available. Aside from the price of current offerings, consumers' skepticism related to the organoleptic properties of the products, and particularly their taste and aroma is a significant limiting factor to widespread acceptance. The present disclosure addresses aspects of consumer appeal by providing compositions and methods that impart, modify or improve organoleptic properties such as taste and aroma, but also colour, texture, mouthfeel and even nutritional value of cultivated meat or comestible products containing them.

For convenience, certain terms employed in the specification, examples, and claims are described herein.

### DEFINITIONS

The term “cultivated meat” is used herein to describe meat that is grown in-vitro in non-human, animal cell culture rather than being obtained from slaughtered animals.

The term “exogenous supplement(s)” refers to an ingredient or mixture of ingredients, which are added to a cell culture medium and/or a scaffold in a nanocarrier, nanoparticle, micellar, liposome or vesicle composition, forming the “supplement composition” or “exogenous supplement composition”, that when contacted with a cell or scaffold are taken-up by, or attached or bound to the cell or scaffold.

The term “comprised in a carrier” as used herein refers to any compounds or molecules that can act as a carrier, for example, without limitation, to nanocarriers, nanoparticles, micelles, liposomes or vesicles as described hereinafter.

The term “nanocarrier(s)” as used herein refers to a nanomaterial being used as a transport means for another substance (i.e., the nanocarrier(s) is a means for carrying or transporting another substance). According to the present disclosure the other substance is an exogenous supplement or cell medium comprising an exogenous supplement. Nanocarriers as used in the present disclosure include, for example, micelles, polymers, carbon-based materials, liposomes and other substances. The nanocarriers as used herein range from sizes of diameter of 1-1000 nm, preferably 1-500 nm and more preferably 1-200 nm.

The term “nanoparticle(s)” as used herein refers to particles in the nano-range having different shape or constitution and ranging from sizes of diameter of 1-1000 nm, preferably 1-500 nm and most preferably 1-200 nm.

Examples for nanoparticles and nanocarriers as used herein include but are not limited to nanomaterials of food-related proteins, such as albumins such as lactalbumin, globulins such as lactoglobulin, glutelins such as glutenin, prolamins such as zein, scleroproteins or protamines, glycoproteins such as avidin, lipoproteins, phosphoproteins or chromoproteins. Nanocarriers as used in the present disclosure explicitly may include virus-like particles or chylomicrons. Other examples are oil-like materials such as, but not limited to, palm oil, sunflower oil or triglycerides, such as, but not limited to, medium chain triglycerides.

The term “micelle(s) or micellar” as used herein refers to spherical aggregates of amphiphilic molecules in which the non-polar molecular regions form a core which are shielded by polar molecular regions in the micellar shell.

The term “liposome(s)” as used herein refers to spherical aggregates having at least one lipid bilayer forming a cavity capable of carrying a liquid. The bilayer is preferably composed of amphiphilic molecules such as, but not limited to, phospholipids, sphingolipids or combinations thereof. The liposome(s) can optionally comprise additives of an aqueous core which is encapsulated by a unilamellar or multilamellar phospholipid shell.

The term “vesicle(s)” as used herein refers to a structure within or outside a cell, consisting of a liquid or medium including buffers enclosed by a lipid layer.

The term ‘spherulites’ as used herein refers to multilamellar vesicles constituted of concentric bilayers, that may be used to encapsulate a suitable exogenous supplement. Within multilamellar vesicles, several hydrophilic and lipophilic layers alternate. Suitable spherulites for use in applications described herein are described in U.S. Pat. No. 5,908,697 and WO 2023/111230, the disclosure of which is incorporated herein by reference.

The term “nanocarrier, nanoparticle, micellar, liposomal or vesicle composition” as used herein refers to a mixture containing one or more different nanocarrier, nanoparticle, micelle, liposome or vesicle structures that encapsulate supplements such as flavours, flavour precursors, colours, colour precursors, bioactive compounds or proteins producing flavours, flavour precursors, nutrients etc., or other additives such as cell medium or food-related additives, including nutraceuticals, texture additives, and mixtures thereof.

The term “meat” when used herein either alone or in phrases such as “meat-like” and “non-human animal-derived meat” refers to meat derived from a non-human animal, including but not limited to mammals, such as farm animals like cows, sheep and pigs, such as wild game like as deer, roe deer, moose; including marsupials such as kangaroo; rodents such as mouse, guinea pig or squirrel; reptiles such as crocodile, turtle or snake; oviparous animals such as poultry and ducks; aquatic animals such as shellfish and fish; and arthropods such as insects.

The term “meat” as used herein may be used, but is not limited to, for products suitable for human consumption and pet consumption.

The term “sensory properties” with regard to meat and/or cultivated meat refers to organoleptic properties, such as colour, smell, taste, aroma and texture, including mouthfeel.

The term “pluripotent stem cells (PSCs)” refers to cells that can propagate indefinitely, as well as give rise to every other cell type in the body, including muscle cells, bone cells and fat cells.

The term “induced pluripotent stem cells (iPSCs)” refers to a type of pluripotent stem cell that can be generated directly from differentiated cells by reprogramming processes well known in the art.

The term “embryonic stem cells (ESC)” refers to a type of pluripotent stem cell derived from blastocyst.

The term “immortalised cell lines” refers to cells that, due to mutations, do not undergo replicative senescence and can be maintained in culture for long periods of time.

The term “primary cells” refers to cells freshly isolated from an animal tissue and grown in vitro.

The term “reprogramming” or “differentiating” refers to conversion of one specific cell type to another. According to certain embodiments of the present invention, reprogramming is the conversion of a somatic cell type, to a pluripotent cell type known as an induced pluripotent stem cell, or iPSC.

### METHOD OF THE INVENTION

Cell compositions and cultivated meat of the present disclosure, as well as comestible products of the same can be prepared according to a method comprising the step of contacting a single type or a plurality of cells with at least one exogenous supplement comprised in a carrier, for example, without limitation, in a nanocarrier, nanoparticle, micellar, liposome or vesicle composition selected from flavour materials, flavour precursors, flavour enhancers, colours and colour precursors and bioactive compounds and other food-related additives or mixtures thereof.

According to certain embodiments, the cells are pluripotent stem cells (PSCs) and/or cells differentiated therefrom.

According to certain embodiments, the PSCs are induced PSCs (iPSCs) reprogrammed from somatic non-human animal cells and/or cells differentiated therefrom.

According to certain embodiments, the PSCs are non-embryonic stem cells (non-ESCs).

According to certain embodiments, the PSCs are embryonic stem cells (ESCs).

According to certain exemplary embodiments, non-genetically modified PSCs are reprogrammed cells produced by a method comprising introducing into at least one cell a combination of: (a) at least one reprogramming mRNA encoding reprogramming factor; and (b) at least one double-stranded microRNA; thereby producing at least one iPSC as described in WO2020230138A1, which publication is incorporated herein by reference.

In certain embodiments, the PSCs are bovine-derived embryonic stem cells (ESCs) and are cultured according to methods described in WO2020230138A1, which published methods are incorporated herein by reference.

According to certain embodiments, the cells are pluripotent stem cells differentiated to muscle cells.

According to certain embodiments, the cells are pluripotent stem cells differentiated to fat cells (adipocytes) and/or their progenitors.

According to certain embodiments, the cells are pluripotent stem cells differentiated to stromal cells (connective tissue) and/or their progenitors.

According to certain embodiments, the cells are pluripotent stem cells differentiated to endothelial cells (blood vessels) and/or their progenitors.

According to certain embodiments, the cells are pluripotent stem cells differentiated to Erythroid/Erythroblast: (haemoglobin containing cells) and/or their progenitors.

According to certain embodiments, the cells are myoblast that are induced to differentiation to myotubes and are cultured according to methods described in WO201916795A1, which published methods are herein incorporated by reference.

According to certain embodiments, the cells are satellite cells differentiated to muscle cells and/or their progenitors.

According to certain embodiments, the cells are selected from the group consisting of muscle cells, fat cells, stromal cells, fibroblasts, pericytes, endothelial cells and/or their progenitors, and combinations thereof.

According to certain embodiments, the non-human animal is selected from the group consisting of bovine, sheep, swine, birds, poultry (such as duck, chicken and turkey), wild game (such as deer, roe deer, or moose), shellfish, fish, insect, reptile (such as crocodile, turtle or snake), rodent (such as mouse, guinea pig or squirrel) and any combinations thereof. Each possibility represents a separate embodiment of the present disclosure.

According to certain exemplary embodiments, the non-human animal is bovine, more particularly of the species Bos taurus.

According to certain exemplary embodiments, the cells comprise a combination of non-human animal-derived cells comprising muscle cells and progenitors thereof; fat cells and progenitors thereof; stromal cells and progenitors thereof; endothelial cells and progenitors thereof, or erythroid/erythroblast and progenitors thereof.

According to certain embodiments, the cells are stromal vascular fraction cells that are isolated from adipose tissue according to the methods described in: “Mehta F, Theunissen R, Post MJ. Adipogenesis from Bovine Precursors. Methods Mol Biol. 2019; 1889:111-125”, which published methods are herein incorporated by reference.

According to certain exemplary embodiments, the cells form Organoids. “Organoids” are self-organizing, self-renewing three-dimensional cellular structures that resemble organs in structure and function. They can be derived from adult stem cells, embryonic stem cells, or induced pluripotent stem cells or mixtures thereof. They contain most of the relevant cell types with a topology and cell-to-cell interactions resembling that of the in vivo tissue.

Organoids may be cultured for example according to the methods described in Kar, S. K. et al (Kar, S. K., Wells, J. M., Ellen, E. D. et al. Organoids: a promising new in vitro platform in livestock and veterinary research. Vet Res 52, 43 (2021)), which methods are incorporated herein by reference.

According to certain embodiments, the cell culture of any one of the above-described embodiments forms cultivated meat. According to certain exemplary embodiments, cultivated meat according to the present disclosure comprises a combination of non-human animal-derived cells comprising muscle cells and progenitors thereof; fat cells and progenitors thereof; stromal cells and progenitors thereof; endothelial cells and progenitors thereof, erythroid/erythroblast and progenitors thereof, organoids and combinations thereof.

In accordance with the method of the present disclosure, the cells are contacted with at least one exogenous supplement comprised in a carrier, for example, without limitation, in a nanocarrier, nanoparticle, micellar, liposomal or vesicle composition. The cells may be growing in a medium.

As used herein, the term “medium” or “media” refers to any liquid made up of a mixture of basic nutrients and complex nutrients that provides the conditions to maintain the cells alive and/or that permit their growth, proliferation and/or differentiation for a desired period of time. Those nutrients can include, but are not limited, to essential and non-essential amino acids, glucose, vitamins, inorganic salts and buffers. Additional supplements can include hormones, proteins, such as, without limitation, albumin, fetuin and transferrin; lipids; cholesterol; growth factors; heparin; and trace elements (minerals), such as selenium.

Examples of media that may be used in the present invention include, without limitation, isotonic media, ready-to-use media or customized media. Ready-to-use media are well described in the literature and are adapted to certain cell types, cell lines and cell banks. Customized media in the present invention may refer to isotonic media or ready to use media that are supplemented with further ingredients, such as growth factors, or the like, that are adapted to provide the best conditions to the cells that are maintained in said media.

As used herein, the term “isotonic medium or media” refers to media that provide the minimum requirements to maintain the cells alive. According to an illustrative embodiment, an isotonic medium does not have any of one or more of the following products: growth factors (such as insulin, EGF, FGF10, noggin, R-Spondin, bioactive proteins, nutrients, such as minerals and vitamins), FBS, antibiotics, or the like.

In further embodiments, the medium may be a ready-to-use medium or a customized medium that provides all the nutrients, growth factors, or the like that the cells need to grow, proliferate and/or differentiate. The expert in the art will understand that the medium (ready to use or customized) will be adapted to the cell lines or cell banks employed, the cell cycle, differentiation stage and other variables generally known in the art. Thus, more than one medium may be used subsequently in accordance with the method of the present disclosure.

According to certain embodiments, a cell culture medium may or may not comprise antibiotics.

Antimicrobial peptides (AMPs) are diverse group of natural proteins present in animals, plants, insects and bacteria. These peptides are part of the defence mechanism of a host from pathogenic organisms and have been found to be an alternative to chemical preservatives.

According to certain embodiments, the medium of the present disclosure further comprises AMPs preventing contamination of the cultured cells. As described hereinabove, these peptides are natural preservatives. AMPs are produced by bacteria present in many types of food since ancient times, such as cheeses, yogurts, and Portuguese fermented meat, and have been shown to be safe for human consumption, and thus approved for use in the food industry.

A commonly used AMP is nisin, a peptide having 3.5 kDa molecular weight, 34 amino acids, positive charge and antimicrobial activity against gram-positive bacteria including Bacilli, Micrococci, Staphylococcus aureus, Listeria monocytogenes and Clostridia and low antimicrobial activity against gram-negative bacteria. Nisin is used for protecting and increasing preservation time of pasteurized cheeses, dairy desserts, canned food, salted meat and sea food. Although nisin has been used in the food industry for several decades, no development of resistant food-spoilage organisms has been detected.

The culture medium will be adapted to the type of cells that are cultured. Examples of different media that are specifically conceived for the different cell types mentioned herein can be found in the literature, for example in Barsh and Cunningham et al. (J Cell Physiol. 1977 July; 92(1):115-28; 1977); Verma et al., (Animal Biotechnology. 2020: 269-29), Palm and Thompson (Nature. 2017 Jun. 7; 546(7657):234-242), Specht, Liz, and S. Scientist. (“An analysis of culture medium costs and production volumes for cultivated meat.” The Good Food Institute: Washington, DC, USA (2020)), Freshney, R. I., (2021. Culture of Animal Cells: A Manual of Basic Technique and Specialized Applications, Culture of Animal Cells), Sigma-Aldrich, E ECACC Cell Culture Laboratory Handbook, Fundamental Techniques in Cell Culture, Laboratory Handbook. ECACC Handbook, WO201914652A1, which disclosed media are herein incorporated by reference.

In addition to the selection of cell types the cell culture media, the bioprocess (i.e. the culture conditions and bioreactor) is important in creating conditions for the large-scale production of cultivated meat. An important aspect of the bioprocess is the design of the bioreactor (or cultivator) as it will control conditions such as temperature, oxygen levels, the rate of delivery of cell culture media to the cells, as well as other important parameters well known to the person skilled in the art. They also enable monitoring of other critical parameters, such as metabolite levels, pH, accumulation of biomass, cell growth and morphology, metabolite profiles, and the like.

Most non-human animal-derived cells (e.g. chicken embryo fibroblast (CEF) cells) are adherent and thus can be grown attached to a support (such as flasks, culture vessel and other culture supports). Also the non-human animal-derived cells may be grown in suspension in suitable suspension support media.

Scaffolds may assist in the growth of the cells, the cell-to-cell interaction and cell proliferation as well as the adhesion to the support, and as such are useful in culturing cells according to methods of the present disclosure. Scaffolds may also facilitate separation of the cells from the medium (Furuhashi, M. et al., 2021. Formation of contractile 3D bovine muscle tissue for construction of millimetre-thick cultured steak. npj Science of Food, 5.)

Any edible scaffold known in the art for use in cultured cells can be used in accordance with methods of the present disclosure, including edible protein scaffolds, edible hydrogel scaffolds, edible polysaccharide scaffolds and the like. According to certain embodiments, the scaffold is derived from plants, algae, fungi and/or microorganisms. According to certain embodiments, the scaffold can be a 2D or a 3D scaffold, as described in Campuzano S. and Pelling A. E. Front. Sustain. Food Syst., 17 May 2019, which disclosed scaffolds are hereby incorporated by reference.

Thus, in certain embodiments of the present disclosure, the cell compositions as defined hereinabove may comprise a scaffold.

There are many prior art references describing various supports and cell suspension cultures, including WO2021102375A1, US20210106032A1 and WO2020243324A1, which disclosed supports and cell suspension cultures are hereby incorporated by reference.

Cells may be grown in bioreactors, for example as described in Madeline, B. et al., (2015. Culturing a duck ES-derived cell line in single-use bioreactors: A rapid, efficient, and cost-effective vaccine manufacturing system based on suspension culture. BioProcess International, 13.), or in large-scale stirred tank bioreactors (STRs) as described in Eibl, R. et al. (2009. Cell and Tissue Reaction Engineering). Similarly, US 2011/0287508 disclose bioreactors and methods of using them to produce tissue engineered products or culture cells. Furthermore, systems and methods for producing cultured food products such as cultivated meat using a plurality of cell culture bioreactors is described in WO2020222239A1, all of which disclosed bioreactors and methods of using the same are herein incorporated by reference.

In certain embodiments of the present invention, the cell compositions as defined hereinabove may be grown in a bioreactor, such as a stirred bioreactor, or on micro-carrier supports in suspension, which provide solid surfaces on which cells can attach and proliferate.

One of the biggest challenges with suspension cell culture is to prevent the sedimentation of micro-carrier supports, such as beads or discs without dramatically exposing cells to shear forces if high agitation speeds need to be applied. Optimal stirring conditions required for micro-carrier systems must be determined experimentally and addressed on a case-by-case basis as will be appreciated by a person skilled in the art. An operating window needs to be determined within which agitation rates allow for sufficient micro-carrier suspension without damaging the cells being cultivated. Bulk liquid mixing must balance the level of hydrodynamic shear.

In one embodiment, the cell growth and morphology as well as metabolite profiles are monitored.

In accordance with the method according to the disclosure, cells may be contacted with said at least one exogenous supplement composition by means generally known in the art. For example, the cells may be brought into contact with the exogenous supplement composition while they are in the cell culture medium by adding a desired amount of the exogenous supplement composition to the medium where the non-human animal-derived cells are cultured. The supplement composition may be added in one dose at a single time-point, or portions of the supplement composition may be added sequentially over time during the cell culture process. Also the enriched media (which is media supplemented with an exogenous supplement composition) may be prepared before it is added to the cells. The culture medium thus obtained is an enriched medium in accordance with the invention.

In other embodiments, cells can be separated from a growth medium (such as a ready to use medium or a customized medium) and then contacted with at least one exogenous supplement composition. Such an exogenous supplement composition may be provided in the required dilution using an appropriate dilution medium, such as an isotonic solution containing preferably only the exogenous supplement, or the exogenous supplement composition. Preferably, said isotonic solution would contain only food grade products, and would be optionally free of growth factors, FBS, vitamins, antibiotics and the like.

When the cells in culture are grown, proliferated or differentiated in the presence of a scaffold the scaffold may be a scaffold that is enriched with one or more exogenous supplements comprised in a carrier, for example, without limitation, in a nanocarrier, nanoparticle, micellar, liposomal or vesicle composition, optionally before the scaffold is used in cell culture. Also the enriched scaffold (that is, scaffold supplemented with an exogenous supplement composition) may be prepared before cells are added to the scaffold. The scaffold thus obtained is an enriched scaffold in accordance with the disclosure.

For example, and without limitation, the at least one exogenous supplement comprised in a carrier, for example, without limitation, in a nanocarrier, nanoparticle, micellar, liposomal or vesicle composition may be contacted with the scaffold for suitable time periods deemed to be appropriate to enrich the scaffold with the supplement composition, for example at least 1 minute, at least 5 minutes, at least 30 min, at least one hour or for at least 10 hours before the scaffold is brought into contact with the cells.

The contact of the at least one exogenous supplement composition with the non-human animal-derived cells can be performed at any stage of cell growth, proliferation or differentiation. The exogenous supplement compositions may also be added to harvested cells, at the end of culture growth.

In certain embodiments of the disclosure, the step contacting the cells with the one or more exogenous supplement compositions can be carried out at times of high cell-division rate. In so doing, a more intense taste, aroma or other organoleptic effect, such as masking of off-notes may be realised.

Cell growth refers to an increase in the total mass of a cell, including both cytoplasmic, nuclear and organelle volume. Cell growth occurs when the overall rate of cellular biosynthesis (production of biomolecules or anabolism) is greater than the overall rate of cellular degradation.

Cell division (or cell proliferation) is the process by which a parent cell divides into two or more daughter cells. The cell division rate varies from cell lines or types to other cells lines or types.

Cell growth and cell proliferation may occur at the same time.

Cells may also differentiate. Cell differentiation is the process in which a cell changes from one cell type to another. Usually, the cell changes to a more specialized type.

In certain embodiments of the disclosure, the step contacting the cells with the one or more exogenous supplement compositions can be carried out during the growth, proliferation and/or differentiation of the cells.

Cell growth proliferation rate may be measured using any known method in the art such as for example using cell division markers as described in Bernard S. et al (Analysis of Cell Kinetics Using a Cell Division Marker Mathematical Modelling of Experimental Data 2003 May; 84(5): 3414-3424), herein incorporated by reference.

In certain embodiments of the disclosure, the step contacting the cells with the one or more exogenous supplement compositions can be carried out at times of low cell-division rate. In so doing, a more intense taste, aroma, or other organoleptic effect, such as masking of off-notes can be realised.

In certain embodiments of the disclosure, the step contacting the cells with the one or more exogenous supplement compositions can be carried out at times of low cell-differentiation rate.

In so doing, a more intense taste, aroma, or other organoleptic effect, such as masking of off-notes can be realised.

In certain embodiments of the disclosure, the step contacting the cells with the one or more exogenous supplement compositions can be carried out at times of high cell-differentiation rate. In so doing, a more intense taste, aroma, or other organoleptic effect, such as masking of off-notes can be realised.

In certain embodiments of the disclosure, the step contacting the cells with the one or more exogenous supplement compositions can be carried out at times of low cell-growth rate. In doing so, a more intense taste, aroma, or other organoleptic effect, such as masking of off-notes can be realised.

In certain embodiments of the disclosure, the step contacting the cells with the one or more exogenous supplement compositions can be carried out at times of high cell-growth rate. In so doing, a more intense taste, aroma, or other organoleptic effect, such as masking of off-notes can be realised.

According to certain exemplary embodiments, the step contacting the cells with the one or more exogenous supplement compositions is done to cells having substantially constant glucose uptake rate (GUR).

According to certain embodiments, the step of contacting the cells with the one or more exogenous supplement compositions can be facilitated using additional steps that promote the incorporation of the exogenous supplement into the cells.

According to certain embodiments, the contacting of the cells with the one or more exogenous supplement compositions is done at least 5 minutes, at least 10 minutes, at least 20 minutes, at least 30 minutes, at least 40 minutes, at least 50 minutes, at least 1 hour, at least 2 hours, at least 5 hours, at least 10 hours, at least 15 hours, at least 20 hours, at least 1 day, at least 2 days, at least 3 day, such as at least 4 days before culturing is completed, although the precise time may vary depending on the cell type and the cell line, and will be determined based on the desired improvement or modification of the taste and aroma.

According to an illustrative embodiment, the step of contacting of cells with the one or more exogenous supplement compositions is done between 30 minutes and 2 days, more preferred between 1 and 24 hours.

In one embodiment, the cells are contacted with the at least one exogenous supplement composition such that at least 30% of the cells contain at least one exogenous supplement, such as at least 40% of the cells, at least 50% of the cells, at least 60% of the cells, at least 70% of the cells, at least 80% of the cells, at least 90% of the cells, at least 95% of the cells such as least 99% of the cells contain at least one exogenous supplement composition.

In one embodiment, the non-human animal-derived cells are contacted with the at least one exogenous supplement composition for at least 5 minutes, at least 10 minutes, at least 20 minutes, at least 30 minutes, at least 40 minutes, at least 50 minutes, at least 1 hour, at least 2 hours, at least 5 hours, at least 10 hours, at least 15 hours, at least 20 hours, at least 1 day, at least 2 days, at least 3 days, such as at least 4 days or at least one week.

According to an illustrative embodiment, the non-human animal cells are contacted with the at least one or more exogenous supplement compositions for at least 30 minutes and up to 2 days, more preferably for at least 1 and up to 24 hours.

The exogenous supplement composition used in accordance with the present disclosure may comprise an exogenous supplement that may be any ingredient suitable for human consumption that can impart, modify or improve the flavour of a substrate to which it is applied.

This may be a material that inherently possesses a desired flavour, or may be a flavour precursor or enhance other flavours. It may be a defined chemical substance or it may be a complex ingredient that is obtained from materials of vegetable, animal or microbiological origin by appropriate physical, chemical, enzymatic or microbiological processes.

Alternatively, the supplement may be a flavour precursor, that is, a material that although it does not possess an inherently desired flavour, it can nevertheless be converted into a material that does possess a desired flavour in response to a suitable stimulus, such as heat treatment, physical treatment, or as the result of a chemical, enzymatic or microbiological process. For example, the flavour precursor may be a mixture of materials, for example, one of which contains amino functionality and another that is a reducing sugar, which can react under thermal stimulus to form a thermal reaction flavour by a complex series of consecutive and/or competing reactions, such as Maillard reactions, Schiff base formation, Strecker degradation, caramelization reactions, and/or other reactions that are beneficial in the development of flavour and/or colour, all of which are well known to the person skilled in the art. Alternatively, metabolites formed in the process of growing, proliferating or differentiating the cells can add desirable flavour or may be precursors of a desirable flavour in response to appropriate stimuli as described hereinabove.

Amino acid/amine sources may be selected from the group consisting of cysteine, methionine, alanine, glycine, lysine, arginine, histidine, tryptophan, proline, valine, glutamic acid, glutamine, aspartic acid, glutathione, other sulphur-containing peptides, HVP (groundnut, soybean wheat/maize gluten), other hydrolysed proteins (for example those that can be derived from milk, egg, fish, blood, liver, bone, collagen), yeast extract, autolysed yeast, meat extract, taurine, pyrrolidone carboxylic acid and combinations thereof.

Reducing sugars are those that either have an aldehyde group or are capable of forming one in solution through isomerism. The aldehyde group allows the sugar to act as a reducing agent in the Maillard reaction, important in the browning of many foods. Cyclic hemiacetal forms of aldoses can open to reveal an aldehyde and certain ketoses can undergo tautomerization to become aldoses. Examples of reducing sugars include, but are not limited to: glucose, fructose, xylose, glyceraldehyde, galactose, lactose, arabinose, maltose, glucose polymers such as starch, hydrolysed starch, and starch-derivatives like glucose syrup, maltodextrin, dextrin, and combinations thereof.

Reaction flavours can produce an abundance of flavour materials useful in the preparation of roasted, savoury, poultry and animalic or meaty notes, including but not limited to ketopiperazines, piperazines, pyrrolizines, pyrazines, sulphides, thiols and maltol derivatives, and mixtures thereof.

Flavours, flavour precursors or flavour enhancers are not intended to be foods as such; they are articles of manufacture that are intended to impart, modify or improve flavour in cultivated meat or in comestibles containing the same. They are essentially non-nutritional, that is, their substantial purpose is to impart flavour, or to enhance, modify or improve the flavour of substrates to which they are added, not to provide nutrition.

Flavours may be selected from ingredients selected from the group consisting of 1-octen-3-ol, 1-Octen-3-one, 2,3-dimethyl Pyrazine, 2,3-pentanedione, 2,4-decadienal, 2,4-nonadienal, 2,4-undecadienal, 2,5-Dimethyl-3-Furanthiol, 2-Acetyl furan, 2-acetyl-2-thiazoline, 2-acetylthiazole, 2-ethyl-3,5-dimethylpyrazine, 2-ethyl-6-methylpyrazine, 2-Furfurylthiol, 2-Methyl tetrahydrofuran-3-one (coffee furanone), 2-Methyl-3-furanthiol, 2-Methyl-3-tetrahydrofuranthiol, 2-Octen-4-one, 2-pentylfuran, 2-tridecanone, 3-mercapto-2-butanone, 4,5-epoxy-2-Decenal, 4-Mercapto-4-methyl-2-pentanone, 5-Methyl Furfural, Acetoin, Acetyl-2-pyrazine, 4-methyl octanoic acid, Alanine, Anserine, Arachidonic Acid, Arginine, bis(2-methyl-3-furyl) disulphide, Butyric Acid, Corylone, Cysteine, Damascenone, Decalactone—delta, Decanoic Acid, dihydroxyacetone, Dimethyl disulphide, Disodium guanylate, Disodium inosinate, Dodecalactone delta, Emoxyfurone, ETHYL 3-METHYL TETRACID; 4-,NAT,1-PG (Maple furanone), Ethyl oleate, Fructose, Furaneol, Furfural, Furfuryl Disulfide, Furfuryl Mercaptan, furfuryl methyl disulphide, gamma-nonalactone, Glucose, Gluthathione, Glycine, Glycogene, Guaiacol, Hexadecanoic Acid (Oleic acid), Hexanal, Hexanoic Acid, Histidine, Indole, isobutyl mercaptan, Isobutyl-4-methyl-5-ethylthiazoline, Isoleucine, isovaleraldehyde, Leucine, linoleic acid, linolenic acid, Lysine, Mercapto-8 Menthene-1 para, Mercapto-Butanone, Methional, Methionine, Methoxy-2-methyl Pyrazine, Methyl-12-tridecanal, Methyl-2-butyric acid, Methyl-2-keto-3-tetrahydrothiophene, Methylmercaptan, nonanal, Phenyl Ethyl Alcohol, Phenyl ethyl mercaptan, phenylacetaldehyde, Phenylalanine, Plasmalogen, Proline, Ribose, Skatol, Sotolone, Succinic Acid, Sulfurol, Tetradecalactone, delta, Thialdine, trans-2-nonenal, 6-nonenal, trans-2-octenal, trans-2-undecenal, Trimethyl Pyrazine, 2,3,5-Nat, Trithioacetone, Trypthophan, Tyrosine, Valeraldehyde, Valine, Xylose and mixtures thereof.

According to certain embodiments, the flavour material, such as a flavour, a flavour precursor or a flavour enhancer, has a C log P of at least −4, such as at least −3, such as at least −2, such as at least −1, such as at least 0, such as at least 1, such as at least 2, such as at least 3, such as at least 4, such as at least 5, such as at least 6.

According to certain embodiments, the flavour or flavour precursor has a C log P between −4 and 6, preferably such as between −3 and 5, more preferably such as between −2 and 4 and most preferably such as between −1 and 3.

Calculated log P (C log P), is a virtual partition coefficient, representing a method of determining the log P non-experimentally as a function of fragmental contribution of virtually each atom of a compound considering different correction factors. In short, experimentally verified log P values of compounds or fragments of compounds are modelled using regression techniques and are added up as a multiplication product of defined correction factors. The C log P can be calculated according to any of the methods known in the art such as the methods disclosed in Klopman G. et al. (Mini-reviews in Medical Chemistry. 2005, 5, 127-133).

According to certain exemplary embodiments, the flavour material, such as a flavour, flavour precursor or a flavour enhancer, may be selected from materials with a C log P of at least −4 to up to 6 (such as one or more of 12-Methyl tridecanal, 2,4-decadienal, 2,4-undecadienal, 2-acetyl-2-thiazoline, 2-pentylfuran, 2-tridecanone, bis(2-methyl-3-furyl) disulphide, Damascenone, Decanoic Acid, Dodecalactone delta, Ethyl oleate, Furfuryl Disulfide, Hexadecanoic Acid (Oleic acid), Isobutyl-4-methyl-5-ethylthiazoline, linoleic acid, linolenic acid, Mercapto-8 Menthene-1 para, nonanal, Trithioacetone, and combinations thereof.

In certain embodiments of the present disclosure, particularly interesting meat-like tastes can be generated using flavours selected from the group consisting of Sulfurol, Methylmercaptan, Methional, isobutyl mercaptan, Hexadecanoic Acid (Oleic acid), Furaneol, Corylone, Acetoin, 2-acetylthiazole, 2-Decenal, 2,6-Nonadienal and 1-octen-3-ol, and combinations thereof.

The present disclosure now discloses that, unexpectedly, contacting cells with a medium and/or with a scaffold comprising one or more flavours comprised in a carrier selected from the group consisting of Thiaidine, Methional, Mercapto-8 Menthene-1 para, Indole, hexanal, Furaneol Acetoin, 4-Mercapto-4-methyl-2-pentanone, 3-mercapto-2-butanone, 2-Octen-4-one, 2-Methyl-3-furanthiol and 2,4-decadienal, 1-octen-3-ol results in cultured cells that are useful in the preparation of cultivated cell composition (such as a cultivated meat) having improved, meat-like taste.

According to certain exemplary embodiments, the flavour material is selected from one or more of Thialdine, Methional, Mercapto-8 Menthene-1 para, Indole, hexanal, Furaneol Acetoin, 4-Mercapto-4-methyl-2-pentanone, 3-mercapto-2-butanone, 2-Octen-4-one, 2-Methyl-3-furanthiol and 2,4-decadienal, and 1-octen-3-ol, or combinations thereof.

The present disclosure now discloses that, unexpectedly, contacting cells with a medium and/or with a scaffold comprising one or more of the following flavour materials comprised in a carrier such as Sulfurol Mercapto-8 Menthene-1 para, Hexanoic Acid, Furfuryl Mercaptan, Furaneol, Butanedithiol 2,3, Acetoin, 2-Methyl tetrahydrofuran-3-one (coffee furanone), 2-Methyl-3-furanthiol and 2-acetylthiazole or combinations thereof, results in cultured cells that are useful in the preparation of cultivated meat having an improved meat-like taste.

The present disclosure now discloses that, unexpectedly, contacting cells with a medium and/or with a scaffold comprising one or more of the following flavour materials Tetradecanoic Acid, Skatol, Methional, 2-undecanal, 2-undecenal, 2-octenal, 2-nonenal, 6-nonenal, 2-Methyl-3-furanthiol, 2-Decenal, 2,4-decadienal, 2,4-nonadienal, 12-Methyl tridecanal results in cultured cells that are useful in the preparation of cultivated meat having improved, meat-like taste.

Flavours used in the preparation or meat-like tastes and aromas may include a sulphur source. Sulphur sources may be selected from the group consisting of hydrogen sulphide, cysteine, cystine, methionine, glutathione, thiamine, inorganic sulphides, organic thiols and sulphides, 2-mercaptoethanol derivatives, e.g. mercaptoacetaldehyde and/or its dimer 2,5-dihydroxy-1,4-dithiane, 5-hydroxy-3-mercaptopentanone, 3-mercaptopropan-1-ol, 4,5-substituted thiazoles, thiocarbonates, thioamides, 2-mercaptoalkoanoic acids/amides, mercaptoalkylamines, aminosulphides, S-acetylmercaptosuccinic acid, vegetable extracts, fermented vegetable juices, yeast extract, autolysed yeast, egg protein, meat extract and combinations thereof.

Certain flavors or compounds intended to be used as exogenous supplements, having regard to certain physicochemical parameters, such as C Log P, molecular weight and functional groups of the exogenous supplement, may be preferentially transported by a particular carrier material or carrier system.

In a preferred embodiment, wherein the carrier is a liposome, the exogenous supplement may be selected from the group consisting of vitamin B12, succinic acid, butyric acid, lactic acid, cysteine, thiamine, 2,4-decadienal, 2-hexanal, corylone and combinations thereof.

In a preferred embodiment, wherein the carrier is a nanoparticle or nanocarrier, the exogenous supplement may be selected from the group consisting of arachidonic acid, 12-methyl tridecanal, linoleic acid, 2-pentylfuran, 4-methyloctanoic acid, 2,4-decandienal and combinations thereof.

In a preferred embodiment, wherein the carrier is a spherulite, as described herein, the exogenous supplement may be selected from the group consisting of vitamin B12, succinic acid, butyric acid, lactic acid, cysteine, thiamine, 2,4-decadienal, 2-hexanal, corylone, arachidonic acid, 12-methyl tridecanal, linoleic acid, 2-pentylfuran, 4-methyloctanoic acid, thialdine, furaneol, disodium guanylate, lactic acid, leucine, methionine, disodium inosate, ribose, xylose, glucose, glutamic acid and combinations thereof.

Yeast extracts can also be used to impart flavour. Yeast extracts may be made up of natural components from the yeast cell: proteins, amino acids, carbohydrates, vitamins and minerals. To produce yeast extract, the contents of the yeast cell are broken down by enzymes and the cell wall is removed. Yeast extract is a food ingredient that contains many taste-providing components among them glutamate and its derivatives. It offers multiple taste-enhancement properties. Yeast extract is a popular source of flavour for a range of savoury food products, particularly when a meaty aroma is required (Ames JM and Elmore JS. 1992. Flavor Fragr. J 7:89-103).

According to certain embodiments, the enriched medium and/or scaffold comprises the yeast extract at a concentration of from about 10 pg/ml to about 5 g/ml. According to certain exemplary embodiments, the enriched medium and/or scaffold comprises yeast extract at a concentration of from about 50 pg/ml to about 1 g/ml. According to certain exemplary embodiments, the enriched medium and/or scaffold comprises yeast extract at a concentration of from about 500 pg/ml 50 mg/ml.

A complete flavour composition may comprise flavour precursors, aroma volatiles and other ingredients used in the creation of flavour that are generally known in the art, such as other synergists or enhancers, including fats or fatty acids, or their sources, herbs, spices and the like; pH regulators; inorganic salts; taste masking agents, taste sensates; vitamins; dyes; colourants; pigments, and the like.

Examples of flavour enhancers and their sources include MSG, IMP, GMP, autolysed yeast, HVP, 2-furfuryl-thioinosine-5′-phosphate, 2-allyloxyinosine-5′-phosphate, 2-(lower alkoxy) inosine-5′-phosphate, 2-benzylthioinosine-5′-phosphate, 4-glucosylgluconic acid, and cyclotene.

Examples of pH regulators include mono-di- and tri-basic inorganic acids, such as HCl, sulphuric acid and phosphoric acid, organic acids, including succinic, citric, lactic, malic, tartaric, acetic and propanoic; amino acids, including valine, glycine and glutamic acids.

Examples of fats include fats of beef, chicken, coconut, other triglycerides, fatty acids, and their esters.

Examples of inorganic salts include chlorides and phosphates.

Taste masking agents might also be employed to mask any off-notes attendant with the basal content of the cultivated meat. Taste masking agents include but are not limited to dihydrochalcones, nucleotides, sodium salts, hydroxyflavanones and the like.

Taste sensates might also be employed. Taste sensates include hot tasting, salivation-inducing substances, substances causing a warm or tingling feeling, and cooling active ingredients. Examples of hot tasting and/or salivation-inducing substances and/or substances which cause a feeling of warmth and/or a tingling feeling on the skin or on the mucous membranes are capsaicin, dihydrocapsaicin, gingerol, paradol, shogaol, piperine, carboxylic acid-N-vanillylamides, for example, without limitation, nonanoic acid-N-vanillylamide, pellitorin or spilanthol, 2-nonanoic acid amides, for example, without limitation, 2-nonanoic acid-N-isobutylamide, 2-nonanoic acid-N-4-hydroxy-3-methoxyphenylamide, alkyl ethers of 4-hydroxy-3-methoxybenzyl alcohol, for example, without limitation, 4-hydroxy-3-methoxybenzyl-n-butylether, alkyl ethers of 4-acyloxy-3-methoxybenzyl alcohol, for example, without limitation, 4-acetyloxy-3-methoxybenzyl-n-butylether and 4-acetyloxy-3-methoxybenzyl-n-hexylether, alkyl ethers of 3-hydroxy-4-methoxybenzyl alcohol, alkyl ethers of 3,4-dimethoxybenzyl alcohol, alkyl ethers of 3-ethoxy-4-hydroxybenzyl alcohol, alkyl ethers of 3,4-methylene dioxybenzyl alcohol, (4-hydroxy-3-methoxyphenyl)acetic acid amides, for example, without limitation, (4-hydroxy-3-methoxyphenyl)acetic acid-N-n-octylamide, vanillo-mandelic acid alkylamides, ferulic acid-phenethylamides, nicotinaldehyde, methylnicotinate, propylnicotinate, 2-butoxyethylnicotinate, benzylnicotinate, 1-acetoxychavicol, polygodial and isodrimeninol.

Hot tasting natural extracts and/or natural extracts which cause a feeling of warmth and/or a tingling feeling on the skin or on the mucous membranes and which can be a constituent of a complete flavour composition include: extracts of paprika, extracts of pepper (for example capsicum extract), extracts of chili pepper, extracts of ginger roots, extracts of Aframomum melegueta, extracts of Spilanthes acmella, extracts of Kaempferia galanga or extracts of Alpinia galangal.

Depending upon the flavour profile that a flavourist is trying to achieve, a complete flavour composition might additionally contain one or more of the following ingredients: dimethyl sulfide, ethyl acetate, ethyl propionate, methyl butyrate, and ethyl butyrate; flavour oils containing volatile aldehydes or esters include, e.g., cinnamyl acetate, cinnamaldehyde, citral, diethylacetal, dihydrocarvyl acetate, eugenyl formate, and p-methylanisole. Further examples of volatile compounds that may be present in the flavour oils include spice oleoresins derived from allspice, basil, capsicum, cumin, dill, garlic, marjoram, paprika, black pepper, rosemary, and turmeric, essential oils, caraway oil, clove oil, onion oil, pepper oil, rosemary oil, spearmint oil, alliaceous flavours, garlic, leek, chive, and onion, botanical extracts, protein hydrolysates, hydrolysed vegetable proteins, meat protein hydrolysates, milk protein hydrolysates and compounded flavours both natural and artificial including those disclosed in S. Heath, Source Book of Flavors, Avi Publishing Co., Westport Connecticut, 1981, pages 149-277; valerian oil; 3,4-dimeth-oxyphenol; amyl acetate; amyl cinnamate, butyryl lactone; furfural; trimethyl pyrazine; phenyl acetic acid; isovaleraldehyde; ethyl maltol; ethyl vanillin; ethyl valerate; ethyl butyrate; cocoa extract; coffee extract; peppermint oil; spearmint oil; clove oil; anethol; cardamom oil; wintergreen oil; cinnamic aldehyde; ethyl-2-methyl valerate; γ-hexenyl lactone; 2,4-decadienal; 2,4-heptadienal; methyl thiazole alcohol (4-methyl-5-β-hydroxyethyl thiazole); 2-methyl butanethiol; 4-mercapto-2-butanone; 3-mercapto-2-pentanone; 1-mercapto-2-propane; benzaldehyde; furfural; furfuryl alcohol; 2-mercapto propionic acid; alkyl pyrazine; methyl pyrazine; 2-ethyl-3-methyl pyrazine; tetramethyl pyrazine; polysulfides; dipropyl disulfide; methyl benzyl disulfide; alkyl thiophene; 2,3-dimethyl thiophene; 5-methyl furfural; acetyl furan; 2,4-decadienal; guiacol; phenyl acetaldehyde; β-decalactone; D-limonene; acetoin; amyl acetate; maltol; ethyl butyrate; levulinic acid; piperonal; ethyl acetate; n-octanal; n-pentanal; n-hexanal; diacetyl; monosodium glutamate; monopotassium glutamate; sulfur-containing amino acids, e.g., cysteine; hydrolyzed vegetable protein; 2-methylfuran-3-thiol; 2-methyldihydrofuran-3-thiol; 2,5-dimethylfuran-3-thiol; hydrolyzed fish protein; tetramethyl pyrazine; propylpropenyl disulfide; propylpropenyl trisulfide; diallyl disulfide; diallyl trisulfide; dipropenyl disulfide; dipropenyl trisulfide; 4-methyl-2-[(methylthio)-ethyl]-1,3-dithiolane; 4,5-dimethyl-2-(methylthiomethyl)-1,3-dithiolane; and 4-methyl-2-(methylthiomethyl)-1,3-dithiolane.

Flavour materials, for example, without limitation, useful in the flavouring of cultivated poultry meat include but are not limited to 2-methylbutanal, methylpyrazine, 2,5-dimethylpyrazine, 2,3-dimethylpyrazine, trimethylpyrazine, 2-methyl-3-furanthiol, 2-acetylpyrrole, furaneol, nor-furaneol, sulfurol, proline-valine diketopiperazine, proline-isoleucine diketopiperazine, 3-methylbutanal, 1,2-dimercapto-ethane, 2-(1-mercaptoethyl) furane, 3-mercapto-2-butanone, 2-mercapto-3-pentanone, 3-mercapto-hexan-4-one, phenylacetaldehyde, 4-methylpentanoic acid, 5-hydroxy-5,6-dihydromaltol, 2-methylthiazolidine, 2-isopropylthiazolidine, 2-isobutylthiazolidine, 2-sec-butyl-thiazolidine, 4,5-dimethylthiazole, 2,5-dimethyl-3-furanthiol, 2-methyl-3-mercaptothiophene, 2-methyl-5-(1-mercaptomethyl)-thiophene, 2-mercaptopro-pionic acid, valine-valine diketopiperazine, valine-alanine diketopiperazine, proline-alanine diketopiperazine and proline-leucine diketopiperazine.

Other ingredients include aldehyde and ketone sources, including acetaldehyde, propanal, butanal, methylpropanal, C3 to C5 alkanals, HVP, alpha diketones and sources thereof, including butanedione, pentane-2,3-dione, pyruvaldehyde, pyruvic acid, glyceraldehyde, glyoxal, dihydroxyacetone, alpha-ketobutyric acid, heptane-3,4-dione-2,5-diacetate, HMFone, HDFone, and related derivatives, ascorbic acid, 5-ketogluconic acid, cyclotene, maltol, lactic acid, glycolic acid, malic acid, tartaric acid, and protein hydrolysates.

Flavour materials, for example, without limitation, useful in the flavouring of cultivated red meats include but are not limited to 2-methylbutanal, 2-methyl-1-butene-1-thiol, 2-methyl-3-furanthiol, 2,5-dimethylpyrazine, 2,6-dimethylpyrazine, 2,3-dimethylpyrazine, 2-methyl-4,5-dihydrofuran-3-thiol, trimethylpyrazine, furfuryl mercaptan, 2-acetylpyrrole, furaneol, nor-furaneol, sulfurol, proline-valine diketopiperazine, proline-isoleucine diketopiperazine, 3-methylbutanal, 3-mercapto-2-butanone, 2-ethyl-8-methylpyrazine, 2-methylthiazolidine, 2-isopropylthiazolidine, 2-isobutylthiazolidine, maltol and proline-leucine diketopiperazine.

Other ingredients include aldehyde and ketone sources, including acetaldehyde, propanal, butanal, methylpropanal, C3 to C5 alkanals, HVP, alpha diketones and sources thereof, including butanedione, pentane-2,3-dione, pyruvaldehyde, pyruvic acid, glyceraldehyde, glyoxal, dihydroxyacetone, alpha-ketobutyric acid, heptane-3,4-dione-2,5-diacetate, HMFone, HDFone, and related derivatives, ascorbic acid, 5-ketogluconic acid, cyclotene, maltol, lactic acid, glycolic acid, malic acid, tartaric acid, and protein hydrolysates.

According to certain embodiments, the exogenous supplement composition is added to the medium and/or to the scaffold in a concentration of from about 100 pg/ml to about 5 mg/ml, such as from about 1000 pg/ml to about 100 ng/ml, such as from about 10 ng/ml to about 100 ng/ml, such as from about 100 ng/ml to about 5 mg/ml, such as from about 100 ng/ml to about 1 mg/ml.

According to some embodiments, the supplement composition is added to the medium and/or to the scaffold to provide a total concentration of from about 5 ng/ml to about 500 ng/ml.

According to some embodiments, the supplement composition is added to the medium and/or to the scaffold to provide a concentration of from about 0.1 pM to about 500 mM, more particularly about 0.1 nM to about 200 nM, and more particularly still about 1 mM.

The values mentioned herein, refer to the amount of a single supplement or in case mixtures of supplements are used, the sum of the mix of supplements in the supplement composition.

In certain embodiments, the cells are further contacted with at least one additional exogenous supplement composition selected from the group consisting of vitamins, minerals, yeast extracts, bioactive compounds, bacterial extracts, colours, nutrients, texture additives and any combination thereof.

A vitamin is an organic molecule (or a set of molecules closely related chemically, i.e. vitamers) that is an essential micronutrient which an organism needs in small quantities for the proper functioning of its metabolism. Essential nutrients cannot be synthesized in the organism, either not at all or not in sufficient quantities, and therefore must be obtained through diet. Vitamins that may be contacted to the cells include: vitamin A (as all-trans-retinol, all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamin), vitamin C (ascorbic acid), vitamin D (calciferol), vitamin E (tocopherol and tocotrienol), and vitamin K (phylloquinone and menaquinone) and combinations thereof.

Colourants are substances that are added or applied to change the colour of a material, in this case the cells. Colourants used in the present disclosure include natural colourants and synthetic colourants. According to certain illustrative embodiment, the colourants are food grade colourants.

As used herein, the term “natural colourant” refers to a pigment that can be found in natural sources, including plants, algae, fungi and the like. It is to be explicitly understood that the natural colourant of the present invention can be derived from the natural source or can be synthesized chemically. Examples of colourants include but are not limited to carotenoids, caramel colours (such as Class IV E150d), caramelised fruits and vegetables juice concentrate, anthocyanin containing colourants, phycobilin containing colourants, betain containing colourants, betalain containing colourants and any mixture thereof.

Anthocyanin may be present as an extract obtained or obtainable from a plant from the Brassicaceae family (such as Raphanus sativus L. (red radish)), the Rosaceae family (such as Fragaria (strawberry)), the Solanaceae family (such as Solanum tuberosum (red potato)), the Convolvulaceae family (such as Ipomoea batatas (purple sweet potato root)), the Apiaceae family (such as Daucus carota ssp. sativus var. atrorubens Alef. (black carrot)) or mixtures thereof.

In one embodiment, the phycobilin may be present as an extract obtained or obtainable from a cyanobacteria from the Arthrospira platensis, A. fusiformis, or A. maxima species. Optionally, the phycobilin may be obtained or obtainable from Arthrospira platensis (spirulina).

In one embodiment, the betalain may be present as an extract obtained or obtainable from a plant from the Amaranthaceae family. Optionally, the plant from the Amaranthaceae family may be Beta vulgaris (beet).

Anthocyanins are glycosides of the sugar-free anthocyanidins (the aglycone). The sugar molecules in anthocyanins are bound via O-glycosidic bonds to one or more of the hydroxy groups typically present in an anthocyanidin molecule. Most naturally occurring anthocyanins are 3-O-glycosides.

In one embodiment, the anthocyanin is a black carrot derived colour.

Phycobilins are light-harvesting pigments found in cyanobacteria, but they are not present in higher plants. The fundamental structure of phycobilins consists of a tetrapyrrole unit, in which the four pyrrole rings form an open chain. There are four major phycobilins in photosynthetic organisms, phycoerythrobilin, phycocyanobilin, phycoviolobilin and phycourobilin. Differences in the extent of π-electron conjugation are responsible for the distinct absorption spectral properties and colouration of the chromophores. Phycoerythrobilin appears red, phycocyanobilin is blue, phycoviolobilin is purple, and phycourobilin is yellow coloured.

In one embodiment, the phycobilin is a spirulina derived colour. Optionally, the phycobilin in the present invention may be phycocyanobilin, which has a blue colour.

Betalains are a class of red and yellow tyrosine-derived pigments found in plants of the order Caryophyllales, where they replace anthocyanin pigments. There are two categories of betalains:
a) Betacyanins, which appear reddish to violet. Examples of betacyanins present in plants include betanin, isobetanin, probetanin, and neobetanin; and
b) Betaxanthins, which appear yellow to orange. Betaxanthins present in plants include vulgaxanthin, miraxanthin, portulaxanthin, and indicaxanthin.

Therefore, the betalains used in the present disclosure may be betacyanins, such as betanin, isobetanin, probetanin, and neobetanin; and/or betaxanthins, such as vulgaxanthin, miraxanthin, portulaxanthin, and indicaxanthin.

In one embodiment, the betalain is a beetroot derived colour. Optionally, the betalain used in the present invention may be betanin.

Carotenoids, also called tetraterpenoids, are yellow, orange, and red organic pigments that are produced by plants and algae, as well as several bacteria, and fungi. Carotenoids give the characteristic colour to pumpkins, carrots, corn, tomatoes, canaries, flamingos, salmon, lobster, shrimp, and daffodils. In one embodiment, the carotenoids are a carrot derived colour. In one embodiment, the carotenoids are a Dunalliela derived colour.

According to illustrative embodiments, the colourants (such as natural colourants) provide a brown, red, pink or orange colour.

According to certain embodiments, the enriched medium and/or the enriched scaffold comprises a colour at a concentration of about 10 pg/ml to about Sg/ml. According to certain exemplary embodiments, the enriched medium and/or the enriched scaffold comprises a colour at a concentration of about 50 pg/ml to about 1 g/ml. According to certain exemplary embodiments, the enriched medium and/or the enriched scaffold comprises a colour at a concentration of about 500 pg/ml to about 50 mg/ml.

The exogenous supplement compositions concentration in the media, scaffold and cells may be measured using the techniques known in the art and which are described in the examples herein below.

According to certain embodiments, the exogenous supplement compositions or the cell compositions and cultivated meat of the present disclosure, as well as comestible products of the same comprise a carrier which can be selected from the group of nanocarriers, nanoparticles, micelles, liposomes or vesicles, preferably from the group of nanoparticles, micelles and liposomes, more preferably the carriers are liposomes, or most preferably the carriers are liposomes composed of phospholipids.

According to certain exemplary embodiments of the invention, the exogenous supplement composition is coloured or is capable of generating colour, thereby its uptake from the culture medium into and/or onto the cell and/or its amount in or on the cell can be measured by a spectrophotometer at a wavelength specific for each supplement and such supplement composition. Minerals include, without limitation, calcium, phosphorus, potassium, sodium, magnesium, sulphur, iron, chlorine, cobalt, copper, zinc, manganese, molybdenum, iodine, and selenium.

Optionally, a method in accordance with the present disclosure may further comprise a step whereby once the cells have incorporated the exogenous supplement composition, any excess supplement or supplement composition is separated from the cells.

In certain embodiments, the cells are partially separated from the enriched medium and/or the enriched scaffold after at least 1 minute, at least 10 min, at least 30 min, at least 1 h of contact with the exogenous supplement composition, at least 2 hours, at least 3 hours, at least, 4 hours, at least 5 hours, at least 10 hours, at least 15 hours, at least 20 hours, at least 24 hours, at least 2 days, at least 2 day or at least one week.

According to certain illustrative embodiments, the cells are partially separated from the enriched medium and/or the enriched scaffold after at least 1 minute to after at least 2 days, more preferred after at least 4 hours to after at least 24 hours.

The total or partial separation of the cells from enriched medium and/or the enriched scaffold may be carried out by any method known in the art such as centrifugation, filtration, decantation, fluorescence-activated cell sorting or isopycnic sedimentation between other methods. Cells can be separated from non-continuous culture or from continuous culture.

In certain embodiments, the at least one exogenous supplement composition is present in the cells at a site selected from the group consisting of the cell intracellular space, intramembrane space, on the membrane and any combination thereof.

In certain embodiments, the cells are separated from enriched medium and/or the enriched scaffold before the exogenous supplement composition is partially or totally metabolized.

According to certain illustrative embodiments, at least 20%, at least 30%, at least 40%, at least 50%, at least 60% of the cells have not partially or totally metabolized the at least one exogenous supplement, such as at least 70%, at least 80%, at least 90%, at least 95% or at least 99% of the non-human animal-derived cells have not partially or totally metabolized the at least one exogenous supplement.

According to certain illustrative embodiments, at least 20%, at least 30%, at least 40%, at least 50%, at least 60% of the cells have not partially or totally metabolized the at least one exogenous supplement composition, such as at least 70%, at least 80%, at least 90%, at least 95% or at least 99% of the non-human animal-derived cells have not partially or totally metabolized the at least one exogenous supplement composition.

The term “metabolize(d)” shall encompass any kind of change of a compound by a chemical reaction, for example, without limitation, by chemical degradation reactions, but shall also include the change in chemical or biological activity. The term metabolite shall encompass the product derived by such reactions or change in activity.

In certain embodiments, the cells are separated from enriched medium and/or the enriched scaffold when at least 30% of the cells contain at least one of the exogenous supplement compositions, such as at least 40% of the cells, at least 50% of the cells, at least 60% of the cells, at least 70% of the cells, at least 80% of the cells, at least 90% of the cells, at least 95% of the cells, at least 99% of the cells contain at least one exogenous supplement composition.

Any method known in the art to measure the amount of the exogenous supplement composition taken up by the cells can be used according to the teachings of the present invention. For example, HPLC analysis of the % of supplements that are incorporated to the cells can be performed according to the examples of the present disclosure. For example, the cells may be centrifuged to precipitate cellular debris and membrane proteins. The centrifugation may be carried out from 200 G to 500 G, such as at 125 G was performed.

Samples may be centrifuged from 1 to 20 minutes, e.g. 15 minutes and the temperature may be 4 °C. The supernatant is then collected and may be dried or the nanocarrier may be disintegrated before being subjected to HPLC analysis.

In certain embodiments, the method further comprises washing the cells in a water-based solution to remove the cell culture medium and exogenous supplement composition that is not taken up by the cells.

In certain embodiments, the cells may be dried with or without separation of the cells from the enriched medium and/or the enriched scaffold of the invention.

Any method known in the art may be used such as centrifugation, filtration, decantation, or the like.

If the cells are grown on a scaffold, a step of disrupting the interaction of the cells with the scaffold surface may be needed to separate the cultivated meat from the scaffold. Various proteolytic enzymes are used to detach cells from the surface of scaffolds, of which trypsin, a member of the serine protease family, is most frequently used. Trypsin is produced from proenzyme, trypsinogen secreted by exocrine cells of pancreas. Trypsin acts on the C-terminal side of Lysine or Arginine. Optimum activity is achieved at 37 °C, so pre-warmed trypsin is capable of accelerating detachment (see for example https://www.siamaaldich.com/CH/de/technical-documents/DrotocoV/cell-culture-and-cell-culture-analvsis/mammalian-cell-culture/cell-dissociation-with-trvDsin).

The cell composition thus obtained may be used directly or may be dried.

The cells obtained may be dried by any known methods in the art such as freeze drying (Zhang et al., 2017 Freeze-drying of mammalian cells using trehalose: preservation of DNA integrity. Scientific reports, 7(1): 6198-6198), drying through microwave processing (Gd et al., 2013 Dry preservation of animal cells: state of the art in microwave processing. Cryo letters, 34: 203-204), oven drying, spray drying, absorption on a solid support or any other techniques known in the art. The drying steps can also represent thermal and physical means for generating a flavour from the flavour precursors or a colourant from the colour precursor.

Cell compositions made according to a method as described herein represents yet another aspect of the present disclosure.

The cell compositions of the present disclosure comprise one type or a plurality of cells, wherein at least 20%, at least 40%, at least 50%, at least 60%, at least 80%, at least 90% of the cells contain at least one exogenous supplement comprised in a carrier, wherein the cell compositions are characterized by having improved sensory properties, such as flavour, substantially similar to whole meat.

The cell composition of the present disclosure is enriched with one or more exogenous supplement compositions. In one embodiment, the cell compositions comprise at least 10% of the cells that contain at least one of the exogenous supplement composition, such as at least 20%, at least 30% at least 40% of the cells, at least 50% of the cells, at least 60% of the cells, at least 70% of the cells, at least 80% of the cells, at least 90% of the cells, at least 95% of the cells, at least 99% of the cells contain at least one of exogenous supplement compositions.

The cell composition of the invention may comprise one type or a plurality of cells. The cell composition of the invention may comprise harvested cells, aggregates of cells, or structure 2D or 3D cultivated meat tissue.

In certain embodiments, the exogenous supplement composition is present in the cells of the cell composition at a site selected from the group consisting of the cell intracellular space, intramembrane space, on the membrane and any combination thereof.

In certain embodiments, the cell composition of the present disclosure further comprises at least one additional exogenous supplement composition wherein the exogenous supplement is selected from the group consisting of vitamins, minerals, yeast extracts, bioactive compounds, bacterial extracts, colours, texture additives and any combination thereof. Examples of vitamins, minerals, yeast extracts, bacterial extracts, colours and texture additives are discussed hereinabove or hereinafter.

In certain embodiments, the cell composition of the present disclosure further comprises at least one additional exogenous supplement composition wherein the carrier is selected from the group of nanocarriers, nanoparticles, micelles, liposomes or vesicles, preferably from the group of nanoparticles, micelles and liposomes, more preferably the carriers are liposomes, most preferably the carriers are liposomes composed of phospholipids.

According to certain embodiments, cell compositions comprise a plurality of cells containing minerals (such as iron or a salt thereof) in an amount from about 0.01 mg/100 g cells to about to 50 mg/100 g cells. According to certain embodiments, the minerals (such as iron or a salt thereof) are present in cell compositions in an amount of from about 0.01 mg/100 g cells to about to 40 mg/100 g cells. According to certain embodiments, the minerals (such as iron or a salt thereof) are present in a cell composition in an amount from about 0.05 mg/100 g cells to about to 5 mg/100 g cells.

According to certain embodiments, the cell compositions further comprise at least one vitamin (such as vitamin D). According to certain embodiments, the cell composition comprises a plurality of cells containing vitamins (such as vitamin D) in an amount of from about 0.01 pg/100 g cells to about 150 pg/100 g cells. According to certain embodiments, vitamins (such as vitamin D) are present in an amount from about 0.1 pg/100 g cells to about 100 pg/100 g cells. According to certain embodiments, the vitamin D amount is from about 0.1 pg/100 g cells to about 15 pg/100 g cells.

In certain or preferred embodiments, the carrier comprising the exogenous supplement, wherein the carrier is preferably selected from a nanocarrier, a nanoparticle, a micelle, a liposome or a vesicle, may have a mean particle size is less than 500 nm, preferably less than 350 nm, more preferably less than 200 nm or between 100 nm to 200 nm. At these mean particle sizes, the carrier comprising the exogenous supplement has suitable size to be taken up or incorporated by the cells.

The term “(mean) particle size” as used herein refers to the particle size of the exogenous supplement composition, as described herein. The particle size may be measured by a skilled person using known methods e.g. dynamic light scattering.

In certain embodiments, the at least one exogenous supplement is a food grade supplement (such as a food grade flavour, flavour precursor, a food grade colourant, a food grade texture additive or the like).

In certain embodiments cell compositions of the present disclosure contain an amount of the at least one exogenous supplement of about 0.01 mg/100 g cells to about 100 mg/100 g cells, more preferably 0.05 mg/100 g cells to about 10 mg/100 g, or even more preferably 0.1 mg/100 g cells to 5 mg/100 g cells.

In certain embodiments, cell compositions of the present disclosure contain an amount of the at least one exogenous supplement composition of about 0.05 mg/100 g cells to about 500 mg/100 g cells, more preferably 0.1 mg/100 g cells to about 50 mg/100 g, or even more preferably 0.5 mg/100 g cells to 25 mg/100 g cells.

In certain embodiments, cell compositions are selected from the group consisting of
a) pluripotent stem cells (PSCs) and/or cells differentiated therefrom,
b) induced pluripotent stem cells PSCs (iPSCs) reprogrammed from somatic non-human animal cells and/or cells differentiated therefrom,
c) embryonic stem cells (ESCs),
d) satellite cells
e) primary precursor cells
and any combination thereof.

In certain embodiments the cells are selected from the group consisting of muscle cells and progenitors thereof; fat cells and progenitors thereof; stromal cells and progenitors thereof; endothelial cells and progenitors thereof; and any combination thereof.

In certain embodiments the cells originate from a non-human animals selected from the group consisting of bovine, sheep, swine, poultry, reptile, rodent, wild game, shellfish, fish and insect and any combination thereof.

The one or more flavour material may be selected from 1-octen-3-ol, 1-Octen-3-one, 2,3-dimethyl Pyrazine, 2,3-pentanedione, 2,4-decadienal, 2,4-nonadienal, 2,4-undecadienal, 2,5-Dimethyl-3-Furanthiol, 2-Acetyl furan, 2-acetyl-2-thiazoline, 2-acetylthiazole, 2-ethyl-3,5-dimethylpyrazine, 2-ethyl-6-methylpyrazine, 2-Furfurylthiol, 2-Methyl tetrahydrofuran-3-one (coffee furanone), 2-Methyl-3-furanthiol, 4-methyl octanoic acid, 2-Methyl-3-tetrahydrofuranthiol, 2-Octen-4-one, 2-pentylfuran, 2-tridecanone, 3-mercapto-2-butanone, 4,5-epoxy-2-Decenal, 4-Mercapto-4-methyl-2-pentanone, 5-Methyl Furfural, Acetoin, Acetyl-2-pyrazine, Alanine, Anserine, Arachidonic Acid, Arginine, bis(2-methyl-3-furyl) disulphide, Butyric Acid, Corylone, Cysteine, Damascenone, Decalactone—delta, Decanoic Acid, dihydroxyacetone, Dimethyl disulphide, Disodium guanylate, Disodium inosinate, Dodecalactone delta, Emoxyfurone, ETHYL 3-METHYL TETR ACID; 4-,NAT,1-PG (Maple furanone), Ethyl oleate, Fructose, Furaneol, Furfural, Furfuryl Disulfide, Furfuryl Mercaptan, furfuryl methyl disulphide, gamma-nonalactone, Glucose, Gluthathione, Glycine, Glycogene, Guaiacol, Hexadecanoic Acid (Oleic acid), Hexanal, Hexanoic Acid, Histidine, Indole, isobutyl mercaptan, Isobutyl-4-methyl-5-ethylthiazoline, Isoleucine, isovaleraldehyde, Leucine, linoleic acid, linolenic acid, Lysine, Mercapto-8 Menthene-1 para, Mercapto-Butanone, Methional, Methionine, Methoxy-2-methyl Pyrazine, Methyl-12-tridecanal, Methyl-2-butyric acid, Methyl-2-keto-3-tetrahydrothiophene, Methylmercaptan, nonanal, Phenyl Ethyl Alcohol, Phenyl ethyl mercaptan, phenylacetaldehyde, Phenylalanine, Plasmalogen, Proline, Ribose, Skatol, Sotolone, Succinic Acid, Sulfurol, Tetradecalactone, delta, Thialdine, trans-2-nonenal, 6-nonenal, trans-2-octenal, trans-2-undecenal, Trimethyl Pyrazine, 2,3,5-Nat, Trithioacetone, Trypthophan, Tyrosine, Valeraldehyde, Valine, Xylose and mixtures thereof.

According to certain embodiments, the exogenous supplement has a C log P of at least −4, such as at least −3, such as at least −2, such as at least −1, such as at least 0, such as at least 1, such as at least 2, such as at least 3, such as at least 4, such as at least 5 or such as at least 6.

According to certain embodiments, the flavour is selected from a flavour, flavour precursor or a flavour enhancer with a C log P of at least −4 to up to 6 (such as one or more of 12-Methyl tridecanal, 2,4-decadienal, 2,4-undecadienal, 2-acetyl-2-thiazoline, 2-pentylfuran, 2-tridecanone, bis(2-methyl-3-furyl) disulphide, Damascenone, Decanoic Acid, Dodecalactone delta, Ethyl oleate, Furfuryl Disulfide, Hexadecanoic Acid (Oleic acid), Isobutyl-4-methyl-5-ethylthiazoline, linoleic acid, linolenic acid, Mercapto-8 Menthene-1 para, nonanal, Trithioacetone.

According to certain embodiments, the flavour is selected from Sulfurol, Methylmercaptan, Methional, isobutyl mercaptan, Hexadecanoic Acid (Oleic acid), Furaneol, Corylone, Acetoin, 2-acetylthiazole, 2-Decenal, 2,6-Nonadienal, 6-nonenal, 1-octen-3-ol and combinations thereof.

According to certain embodiments, the flavour is selected from one or more of Thialdine, Methional, Mercapto-8 Menthene-1 para, Indole, hexanal, Furaneol Acetoin, 4-Mercapto-4-methyl-2-pentanone, 3-mercapto-2-butanone, 2-Octen-4-one, 2-Methyl-3-furanthiol and 2,4-decadienal, 1-octen-3-ol.

According to certain embodiments, the flavour is selected from one or more of Sulfurol Mercapto-8 Menthene-1 para, Hexanoic Acid, Furfuryl Mercaptan, Furaneol, Butanedithiol 2,3, Acetoin, 2-Methyltetrahydro furan-3-one (coffee furanone), 2-Methyl-3-furanthiol and 2-acetylthiazole.

According to certain embodiments, the flavour is selected from one or more of Tetradecanoic Acid, Skatol, Methional 2-Undecenal, 2-undecenal, 2-octenal, 2-nonenal, 6-nonenal, 2-Methyl-3-furanthiol, 2-Decenal, 2,4-decadienal, 2,4-nonadienal, 12-Methyl tridecanal.

In certain embodiments, at least one exogenous supplement is not metabolized in at least 60% of the cells, at least 70%, at least 80%, at least 90%, at least 95% or at least 99% of cells.

In certain embodiments, at least one exogenous composition is not metabolized in at least 60% of the cells, at least 70%, at least 80%, at least 90%, at least 95% or at least 99% of cells.

In certain embodiments of the present disclosure, the uptake of exogenous supplement in cell composition is about 0.01 mg/100 g cells to about 100 mg/100 g cells, preferably 0.05 mg/100 g cells to about 10 mg/100 g cells.

In certain embodiments of the present disclosure, the uptake of exogenous supplement composition in cell composition is about 0.05 mg/100 g cells to about 500 mg/100 g cells, preferably 0.1 mg/100 g cells to about 50 mg/100 g cells.

In certain embodiments the present disclosure provides cultivated meat, such as cultivated poultry meat or cultivated beef meat, comprising a cell composition as herein defined.

In certain embodiments the present disclosure provides comestible compositions comprising a cell composition as defined herein, in admixture with other food grade ingredients.

Cell compositions of the present disclosure may be utilised in the preparation of cultivated meat, or comestible products containing the same.

The cell composition in the form of a slurry of cells may be further processed, for example, by drying and/or extrusion, and optionally mixed with food grade ingredients, such as meat analogues, e.g. vegetable proteins, and other food grade excipients to form comestibles such as nuggets, minced meat, sausage, burgers and other processed meat products. Alternatively, the cell composition may be in the form of structured 2D or 3D whole cuts of cultivated meat. Still further, the cell composition may be used as a flavour ingredient for example in bouillon cubes and fonds, or may be used in the form of a powder or slurry that is intended to be sprayed as a coating onto savoury snacks.

In certain embodiments, the cell composition may be used in comestible products in a concentration of at least 0.1% w/w, at least 1%, at least 2%, at least 5%, at least 10%, at least 15%, at least 20%, at least 25%, at least 30%, at least 35%, at least 40%, at least 50% at least 60%, at least 70%, at least 80%, at least 90% or at least 99% w/w based on the weight of the comestible product.

Food encompasses the following general food categories, as defined by the Food and Drug Administration (FDA): baked goods and baking mixes, including all ready-to-eat and ready-to-bake products, flours, and mixes requiring preparation before serving; beverages, alcoholic, including malt beverages, and cocktail mixes; beverages and beverage bases, non-alcoholic, including only special or spiced teas, soft drinks, coffee substitutes, and fruit and vegetable flavoured gelatin drinks; cheeses, including curd and whey cheeses, cream, natural, grating, processed, spread, dip, and miscellaneous cheeses; chewing gum, including all forms; coffee and tea, including regular, decaffeinated, and instant types; condiments and relishes, including plain seasoning sauces and spreads, olives, pickles, and relishes, but not spices or herbs; confections and frostings, including candy and flavoured frosting, marshmallows, baking chocolate, and brown, lump, rock, maple, powdered, and raw sugars; toppings, and other non-dairy products; egg products, including liquid, frozen, or dried eggs, and egg dishes made therefrom, i.e., egg roll, egg foo young, egg salad, and frozen multicourse egg meals, but not fresh eggs; fats and oils, including margarine, dressings for salads, butter, salad oils, shortenings and cooking oils; fish products, including all prepared main dishes, salads, appetizers, frozen multicourse meals, and spreads containing fish, shellfish, and other aquatic animals, but not fresh fish; fresh eggs, including cooked eggs and egg dishes made only from fresh shell eggs; fresh fish, including only fresh and frozen fish, shellfish, and other aquatic animals; fresh meats, including only fresh or home-frozen beef or veal, pork, lamb or mutton and home-prepared fresh meat-containing dishes, salads, appetizers, or sandwich spreads made therefrom; fresh poultry, including only fresh or home-frozen poultry and game birds and home-prepared fresh poultry-containing dishes, salads, appetizers, or sandwich spreads made therefrom; pastas, including macaroni and noodle products, rice dishes, and frozen multicourse meals, without meat or vegetables; gravies and sauces, including all meat sauces and gravies, and tomato, milk, buttery, and specialty sauces; herbs, seeds, spices, seasonings, blends, extracts, and flavourings, including all natural and artificial spices, blends, and flavours; meat products, including all meats and meat containing dishes, salads, appetizers, frozen multicourse meat meals, and sandwich ingredients prepared by commercial processing or using commercially processed meats with home preparation; milk, whole and skim, including only whole, low-fat, and skim fluid milks; milk products, including flavoured milks and milk drinks, dry milks, toppings, snack dips, spreads, weight control milk beverages, and other milk origin products; plant protein products, including the National Academy of Sciences/National Research Council “reconstituted vegetable protein” category, and meat, poultry, and fish substitutes, analogues, and extender products made from plant proteins; poultry products, including all poultry and poultry-containing dishes, salads, appetizers, frozen multicourse poultry meals, and sandwich ingredients prepared by commercial processing or using commercially processed poultry with home preparation; all commercially processed vegetables, vegetable dishes, frozen multicourse vegetable meals, and vegetable juices and blends; snack foods, including chips, pretzels, and other novelty snacks; soups, home-prepared, including meat, fish, poultry, vegetable, and combination home-prepared soups; soups and soup mixes, including commercially prepared meat, fish, poultry, vegetable, and combination soups and soup mixes.

In one embodiment, the cell composition of the present disclosure is added to a meat comestible, including fish, poultry or to a vegetable comestible, such as for example but without limitation, a meat analogue, to increase the sensory properties of the product (such as to create modify or improve the flavour, a colour, texture, and/or mouthfeel of the comestible. In one embodiment, the cell composition may be added to the comestible to provide a beef flavour.

In certain embodiments, the cell composition may be added to the comestible to create, modify or improve a chicken flavour.

The following examples are presented to more fully illustrate some embodiments of the present disclosure. They should not be construed, however, as limiting the scope of the present disclosure in any manner whatsoever. One skilled in the art can readily devise many variations and modifications of the principles disclosed herein without departing from the scope of the invention.

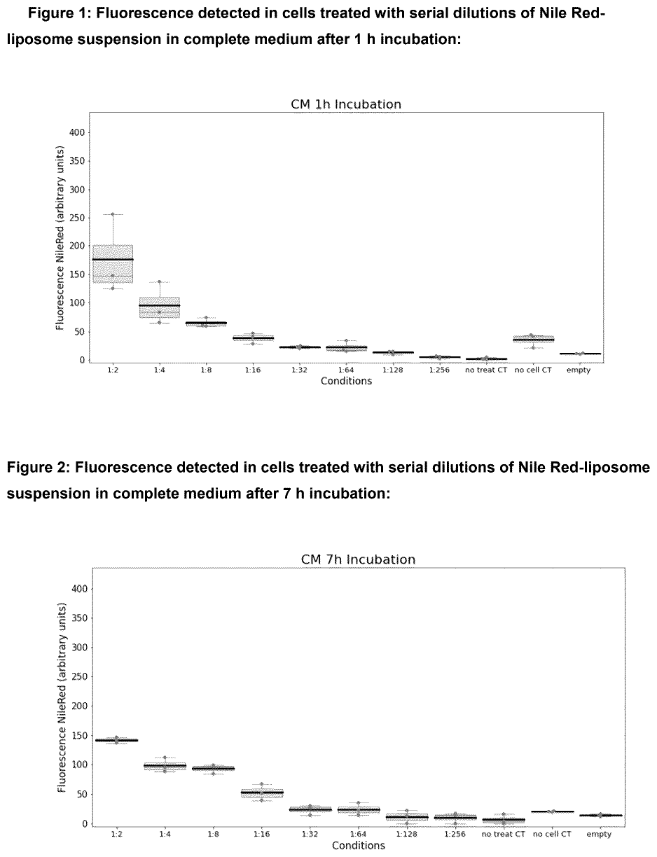

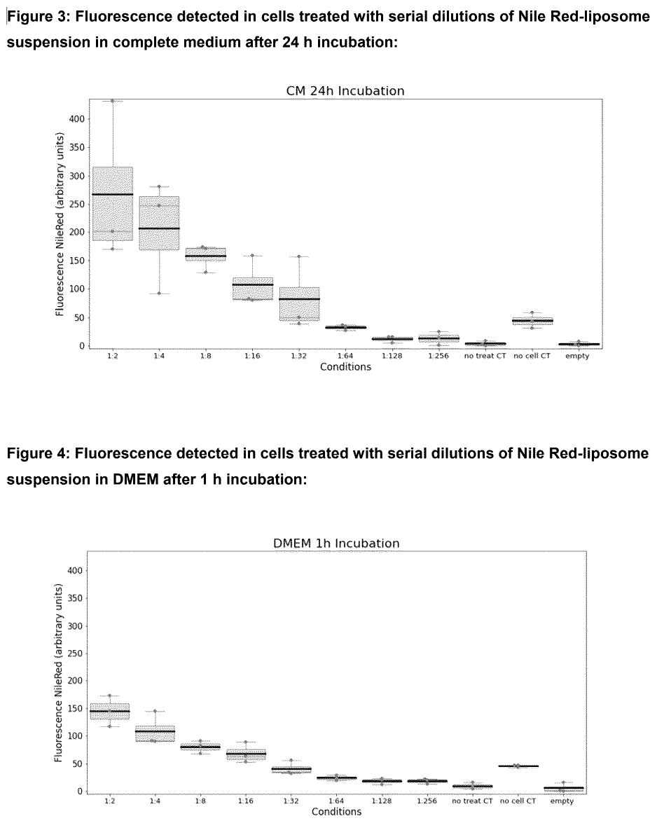

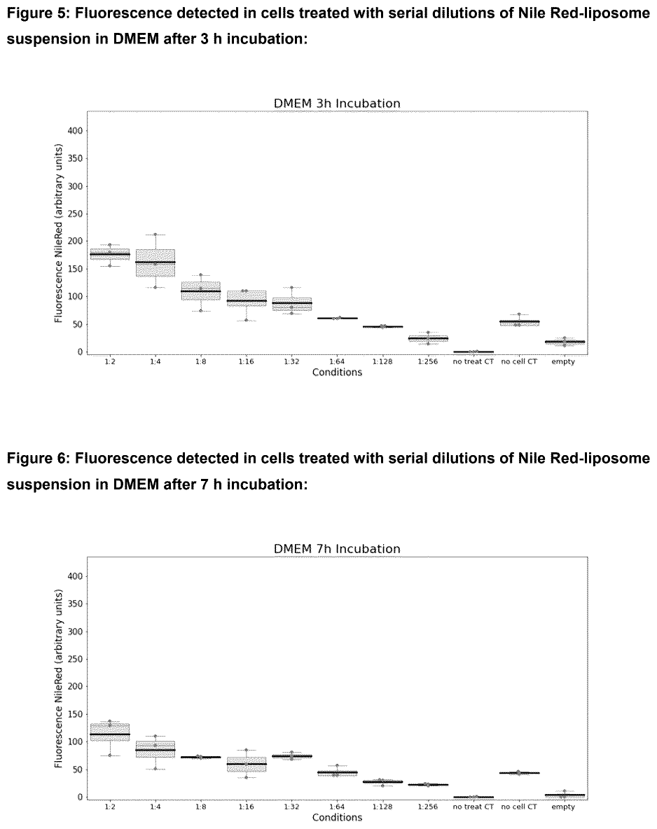

FIGS. 1-3 show fluorescence intensity originating from C2C12 cells cultured in complete medium and incubated with Nile Red loaded liposomes for 1, 7 and 24 hours. FIG. 4-6 similarly show fluorescence intensity originating from C2C12 cells cultured in DMEM and incubated with Nile Red loaded liposomes for 1, 3 and 7 hours. Due to thorough washing of the cells after incubation with the fluorescent liposomes, it can be concluded that fluorescent signals originate from cell-adhered or intracellular fluorescent material. Measurements of fluorescence were always performed under equal measurement protocols (e.g. exposure time, excitation wavelength, gain-settings), making it possible to make comparisons between figures. Increasing incubation time and increased liposomal concentration added to the cell cultures both lead to an increase in measured fluorescence intensity. It is shown that uptake of fluorescent material is possible in serum and serum-free conditions (CM and DMEM, respectively).

### EXAMPLES

1. Liposomes
   1.1. General manufacturing procedure for obtaining liposomes
   1.2. Liposome loading with fluorescence marker
   1.3. Liposome loading with different model compounds
      1.3.1. Flavour
      1.3.2. Colourant
   1.4. Liposome stability study
2. Flavour uptake into cells—Cell preparation
   2.1. Growth conditions
   2.2.1 Liposome uptake—serial dilution
   2.2.2 Liposome uptake—cell imaging
   2.2.3 Liposome uptake and retention/metabolization of (liposome encapsulated) exogenous compounds in cells
   2.2.4 Viability of cells incorporating (liposome encapsulated) exogenous compounds
3. Use of non-human animal cells derived from oviparous animals
4. Use of non-human animal cells derived from stem-cells harvested from umbilical cord, bone marrow, placenta or other tissue
5. Nanoparticles
   5.1 Preparation of flavour-loaded nanoparticles
   5.2 Stability data for flavour-loaded nanoparticles

### Materials

Distearoylphosphatidylcholine (DSPC) was purchased from Avanti Lipids (Birmingham, AL, USA).
Cholesterol was purchased from Merck & Cie. (Schaffhausen, Switzerland).
Nile Red was purchased from Merck & Cie. (Schaffhausen, Switzerland).
Curcumin was provided by Naturex SA (Givaudan SA, Switzerland).
Quinine was purchased from Merck & Cie. (Schaffhausen, Switzerland).
Dulbecco's Modified Eagle Medium (DMEM) with sodium pyruvate and L-Glutamine was purchased from ThermoFisher Scientific (Waltham, MA, USA).
Dulbecco's phosphate-buffered saline (DPBS) was purchased from ThermoFisher Scientific (Waltham, MA, USA).
DMEM plus: DMEM with 4.5 g/L glucose, with sodium pyruvate and L-Glutamine was purchased from Gibco—ThermoFisher Scientific (Waltham, MA, USA) and supplemented with a solution of 1% antibiotic antimycotic (i.e. a solution of Penicillin/Streptomycin/Amphotericin B) which was purchased from Merck & Cie. (Schaffhausen, Switzerland).
Complete Medium (CM): DMEM plus supplemented with 10% Fetal Bovine Serum (FBS) qualified for the US purchased from Biowest (Riverside, MO, USA).
Cell lines can be obtained from one of the suppliers listed in the following publication https://www.labome.com/method/Cell-Lines-Companies.html, or initiated by one of the known methods such as Freshney's Culture of Animal Cells: A Manual of Basic Technique and Specialized Applications. A Capes-Davis, R I Freshney—2021, Choi et al., 2021. comprehensive reviews in food science and food safety. Volume 20, Issue 1. Pages: 1-1117. January 2021, Mehta et al., 2019, Adipogenesis from Bovine Precursors. Methods Mol Biol, 2019; 1889:111-125.
C2C12 cells, a mouse myoblast cell line, were purchased from ECACC (European Collection of Authenticated cell Cultures) through (Merck & Cie, Schaffhausen, Switzerland.
96 well plate with clear bottom and black side walls were purchased from ThermoFisher Scientific (Waltham, MA, USA).
Palm oil was provided by Givaudan Schweiz AG (Dubendorf, Switzerland).
Medium chain triglycerides (MCT) was provided by Givaudan Schweiz AG (Dubendorf, Switzerland).
Soy Lecithin was purchased from Lipoid AG (Steinhausen, Switzerland).
Polysorbate 80 was provided by Givaudan France Naturals (Avignon, France).
All other chemicals used were commercially available reagents or chemical-grade products.

### Example 1—Liposomes

1.1 General Manufacturing Procedure for Obtaining Liposomes with and without an Exogenous Compound for Cell Delivery.

Liposomes were constructed by the lipid film hydration method, in particular:

Step 1a: After acclimatizing to room temperature phospholipids (e.g. lecithins or distearoylphosphatidylcholine) and cholesterol were mixed with the solvent (e.g. a 70:30 v/v mixture of dichloromethane DCM and methanol MeOH).

About 20 mg total solid mass in different molar ratios of 50:50 to 100:0 of phospholipid:cholesterol were placed in a flask and mixed in 50 to 100 mL of the solvent (e.g. chloroform:methanol, 2:1).

Step 1b: Depending on the intended function of the liposomes, solution of an exogenous compound is added to the dissolved lipids in an exogenous compound to lipid weight ratio of 1:500 to 1:1. The addition of exogenous compounds at this stage is preferably for hydrophobic compounds (e.g. c Log P>2).

Step 1c: The mixture was stirred and agitated at room temperature until a clear solution is obtained.

Step 2: A lipid film of liposomes was formed on the wall of the flask by evaporation of organic solvents in a rotavapor at about 30 °C-40 °C and at a rotation speed of 60-100 rpm by first reducing the pressure to 300 mbar and then continuing to reduce the pressure slowly to about 10 mbar to avoid violently boiling. The resulting thin opaque lipid films are flushed with nitrogen for a minute and then thoroughly dried overnight in a vacuum oven at 10 mbar-100 mbar and 40 °C.

Step 3: The lipid film is then prepared for further use by adding 5 mL of pre-warmed water or 5 mL of a relevant buffer (e.g. PBS buffer, pH 7.4) and heating the flask in a water bath, pressurized and at a temperature of 5 °C above the phase transition temperature of the lipids, all at a rotation speed of 50 rpm. The lipid layer is hydrated for at least 1 hour and then kept at 4 °C for the further experiments. In case of encapsulation of exogenous compounds having sufficient water solubility, they may be added at step 3 to the hydration buffer.

Step 4: For forming unilamellar vesicles with a narrow size distribution, extrusion is utilized. Extrusion has been carried out in an Avanti Mini-Extruder, equipped with a polycarbonate membrane with a 100 nm pore size. The extruder is actuated at least 10 times at a temperature of 5 °C above the phase transition temperature of the lipids being processed (e.g. 65 °C).

After extrusion, the liposomes had a median diameter of less than 200 nm. The extruded liposomes may then be stored at 4 °C until further use. If required, unencapsulated exogenous compound may be removed from the samples by using PD Miditrap G-25 gel filtration columns (Cytiva, Grens Switzerland). The samples are then subsequently filtered through 220 nm sterile filters.

1.2 Liposome Loading with a Fluorescence Marker

Different liposomes were prepared according to the general manufacturing process laid out in step 1a. including step 1b, the fluorescence marker was:

Nile Red, 108 μg, added as 108 μL of 1 mg/mL solution in acetone to the lipid/cholesterol solution.

The liposomes containing encapsulated fluorescent markers were purified by passing the formulation through a PD Minitrap desalting column containing Sephadex G-25 resin (Cytiva, commercially available from cytivalifesciences.com, Switzerland).

Separation based on size exclusion resulted in separation of the fluorescently labelled liposomes and unencapsulated fluorescent markers. Fluorescent marker encapsulation in the liposomes was confirmed by measuring fluorescence with a fluorescence microplate reader (Synergy H1 Hybrid Microplate Reader, BioTek, Agilent Technologies, Santa Clara, CA, USA) after column filtration.

1.3 Liposome Loading with Different Model Compounds
1.3.1 Liposome Loading with Flavour Compounds

All experiments and characterization were done in three separate liposome preparations.

52.2 mg Soy Lecithin and 7.8 mg of cholesterol were weighed in a 50 mL round bottom flask and dissolved in 5 mL Chloroform:Methanol (70:30). A meat flavour, 2,4-decadienal, stock solution was made by solving 120 mg 2,4-decadienal in 10 mL pure ethanol. From the 2,4-decadienal stock, 83 μL was added to the soy lecithin and cholesterol solution, resulting in 1 mg of 2,4-decadienal added to the dissolved lipids. The mixture was dried by rotary evaporation at 30 °C under reduced pressure to obtain a dry lipid film. The lipid film was further dried for 4 hours in a vacuum oven at 40 °C. Next, 5 mL of phosphate buffered saline (PBS), pre-warmed at 65 °C, was added to the thin film and the round bottom flask was agitated in a water bath at 65 °C for 1 hour. During hydration, the mixture was vortexed for 1 minute to expedite hydration. The hydrated lipid mixture was extruded in a mini-extruder (Avanti®, Polar Lipids) assembled with a 100 nm mesh polycarbonate membrane. The resulting liposomes were analyzed by dynamic light scattering (Zetasizer nano ZS90, Malvern), showing an average diameter of 142.1 t 3.2 nm and a polydispersity index of 0.090±0.011, indicating narrow diameter distribution. The liposomes were filtered in PD MiniTrap desalting columns with Sephadex G-25 resin (Cytiva commercially available from cytivalifesciences.com, Switzerland) to remove un-encapsulated flavour compounds. Phospholipid concentration in the filtered liposome suspension was analyzed by Stewart assay (John Charles Marshall Stewart, Colourimetric determination of phospholipids with ammonium ferrothiocyanate, Analytical Biochemistry, Volume 104, Issue 1, 1980, Pages 10-14).

Briefly, a reagent solution containing 0.1M FeCl₃ and 0.4MNH₄SCN was made. 50 μL of liposome sample were added to 2 mL reagent solution, 0.45 mL H₂O and 3 mL of chloroform. The mixture was vortexed and centrifuged to separate the aqueous and organic phases. The lower chloroform layer was removed by Pasteur pipette and analyzed for absorption at 485 nm. The liposomal sample was compared to a standard curve of soy lecithin phospholipid solutions with a known concentration. The filtered liposome sample in this example contained 6.3±2.4 mg/mL phospholipids.

The purified liposomes were diluted 10-fold in pure ethanol to disrupt the liposomal membranes and flavour concentration was measured by UV absorption at 282 nm.

The liposomal suspension prepared in this example contained 26.8±2.5 μg/mL 2,4-decadienal. Comparison with unfiltered liposomes (34.0±3.2 μg/mL, including un-encapsulated 2,4-decadienal) showed that the encapsulation percentage was 78.7%±2.8%. In table 1 below, the data from each individual batch can be seen.

TABLE 1
Liposome loading with flavour compounds

| | | | [Soy | 2,4- | EE % 2,4- |
|---|---|---|---|---|---|
| | Diameter | Polydispersity | Lecithin] | decadienal | decadienal |
| Batch | (nm) | Index (PDI) | (mg/mL) | (μg/mL) | (μg/mL) |
| 1 | 143.9 | 0.076 | 5.1 | 27.1 | 74.8% |
| 2 | 137.6 | 0.091 | 4.1 | 23.5 | 79.9% |
| 3 | 144.8 | 0.103 | 9.6 | 29.5 | 81.5% |
| Average ± | 142.1 ± | 0.090 ± | 6.3 ± | 26.8 ± | 78.7% ± |
| SD | 3.2 | 0.011 | 2.4 | 2.5 | 2.8% |

1.3.2 Liposome Loading with Colouring Compounds

Different liposomes were prepared according to the general manufacturing process laid out in step 1a. including step 1b, the different model compounds markers were: Curcumin, 100 μL of a 1 mg/mL solution in acetone to the lipid/cholesterol solution. Quinine, 100 μL of a 1 mg/mL solution in ethanol to the lipid/cholesterol solution. Quinine is a fluorescent flavour material. Fluorescence microscopy and fluorescence microplate readings are used to quantify intracellular quinine levels.

The liposomes containing encapsulated quinine and curcumin were purified by passing the formulation through a PD Minitrap desalting column containing Sephadex G-25 resin (Cytiva, commercially available from cytivalifesciences.com, Switzerland). Separation based on size exclusion resulted in separation of the fluorescently labelled liposomes and unencapsulated fluorescent markers. Fluorescent marker encapsulation in the liposomes was confirmed by measuring fluorescence with a fluorescence microplate reader (Synergy H1 Hybrid Microplate Reader, BioTek, Agilent Technologies, Santa Clara, CA, USA) after column filtration.

Liposome loading was quantified by disrupting the liposomes in Ethanol (100 μL liposome suspension in 900 μL ethanol) and measuring the concentration of colouring compound by UV/VIS absorption or HPLC analysis UV absorption of quinine peaks at 350 nm, fluorescence absorption at 480 nm, while curcumin UV/VIS absorption peaks around 420 nm.

### 1.4 Liposome Stability Study

The stability of liposomes prepared in PBS under storage conditions (4 °C) were followed over 28 days (Table 2). Liposomes with a 20 molar percentage of cholesterol showed excellent size stability under these storage conditions with minimal changes in average diameter and polydispersity (size distribution). Liposomal stability under cell culture mimicking conditions (diluted in DMEM, 37 °C) were followed over 14 days (Table 3). The internalized aqueous phase of the liposomes in DMEM contains PBS.

TABLE 2
Average diameter measurements (Ø) and polydispersity index (PDI) of liposomes
with varying cholesterol content, done by dynamic light scattering (DLS)

| | Day 0 | Day 1 | Day 3 | Day 7 | Day 14 | Day 28 |
|---|---|---|---|---|---|---|
| | A. Ø | A. Ø | A. Ø | A. Ø | A. Ø | A. Ø |
| Conditions | B. PDI | B. PDI | B. PDI | B. PDI | B. PDI | B. PDI |
| Soy Leci- | A. 183.6 | A. 181.3 | A. 167.9 | A. 163.7 | A. 169.1 | A. 167.8 |
| thin:Choles- | B. 0.080 | B. 0.076 | B. 0.049 | B. 0.084 | B. 0.062 | B. 0.055 |
| terol 70:30, | | | | | | |
| PBS, 4 °C | | | | | | |
| Soy Leci- | A. 152.0 | A. 151.2 | A. 153.1 | A. 152.4 | A. 152.5 | A. 154.5 |
| thin:Choles- | B. 0.068 | B. 0.063 | B. 0.064 | B. 0.054 | B. 0.074 | B. 0.060 |
| terol 80:20, | | | | | | |
| PBS, 4 °C | | | | | | |
| Soy Leci- | A. 133.7 | A. 136.7 | A. 138.8 | A. 141.0 | A. 141.2 | A. 141.1 |
| thin:Choles- | B. 0.055 | B. 0.046 | B. 0.026 | B. 0.051 | B. 0.070 | B. 0.052 |
| terol 90:10, | | | | | | |
| PBS, 4 °C | | | | | | |
| Soy Leci- | A. 139.5 | A. 140.4 | A. 131.0 | A. 134.3 | A. 134.8 | A. 134.0 |
| thin:Choles- | B. 0.043 | B. 0.057 | B. 0.035 | B. 0.067 | B. 0.076 | B. 0.056 |
| terol 95:5, | | | | | | |
| PBS, 4 °C | | | | | | |
| Soy Leci- | A. 132.5 | A. 128.0 | A. 133.4 | A. 133.6 | A. 133.8 | A. 134.7 |
| thin:Choles- | B. 0.068 | B. 0.072 | B. 0.084 | B. 0.054 | B. 0.054 | B. 0.042 |
| terol 100:0, | | | | | | |
| PBS, 4 °C | | | | | | |

TABLE 3
Average diameter measurements (Ø) and polydispersity
index (PDI) of liposomes with a lipid:cholesterol molar
ratio of 80:20, done by dynamic light scattering (DLS)

| | Day 0 | Day 1 | Day 2 | Day 5 | Day 7 | Day 14 |
|---|---|---|---|---|---|---|
| | A. Ø | A. Ø | A. Ø | A. Ø | A. Ø | A. Ø |
| Conditions | B. PDI | B. PDI | B. PDI | B. PDI | B. PDI | B. PDI |
| Soy Leci- | A. 152.0 | A. 138.3 | A. 139.3 | A. 138.4 | A. 148.4 | A. 138.2 |
| thin:Choles- | B. 0.068 | B. 0.197 | B. 0.177 | B. 0.189 | B. 0.193 | B. 0.173 |
| terol 80:20, | | | | | | |
| DMEM, 4 °C | | | | | | |
| Soy Leci- | A. 152.0 | A. 138.7 | A. 139.5 | A. 142.3 | A. 140.7 | A. 136.4 |
| thin:Choles- | B. 0.068 | B. 0.168 | B. 0.170 | B. 0.166 | B. 0.159 | B. 0.156 |
| terol 80:20, | | | | | | |
| DMEM, 37 °C | | | | | | |

Further exemplary exogenous compounds encapsulated by liposomes prepared by the methods described herein and associated stability data, determined by dynamic light scattering, is described in table 3.1:

TABLE 3.1
Average diameter measurements (Ø) and polydispersity index (PDI) of liposomes
with encapsulated exogenous compound, done by dynamic light scattering (DLS)

| | | Day 0 | Day 1 | Day 2 | Day 4 |
|---|---|---|---|---|---|
| | Exogenous | A. Ø | A. Ø | A. Ø | A. Ø |
| Conditions | compound | B. PDI | B. PDI | B. PDI | B. PDI |
| DSPC:Choles- | Cobalamin | A. 195.2 | A. 188.9 | N/M | N/M |
| terol 50:50, | (Vitamin | B. 0.128 | B. 0.066 | | |
| PBS, 4 °C | B12) | | | | |
| DSPC:Choles- | Thiamine | A. 155.3 | A. 157.1 | N/M | A. 142.3 |
| terol 50:50, | HCl | B. 0.087 | B. 0.048 | | B. 0.166 |
| PBS, 4 °C | | | | | |
| DSPC:Choles- | Corylone | A. 136.4 | N/M | A. 140.4 | N/M |
| terol 80:20, | | B. 0.082 | | B. 0.072 | |
| PBS, 4 °C | | | | | |

N/M = not measured

### Example 2—Flavour Uptake into Cells—Cell Preparation

### 2.1 Growth Conditions

All of the following experiments are performed in triplicate.

2.2.1 Liposome Uptake into Growing Cell Cultures—Serial Dilution.

C2C12 cells were seeded into a 96 multi-well plate with clear bottom and black side walls at 3000 cells/well (200 μl per well) in CM. Growth media (DMEM) (5 mL) with 5-20% (v/v) FBS was added to the wells, and the cells were cultured until approximately 70% confluency with a homogeneous distribution (cell culture stage 1). A serial dilution of liposomes was prepared in the different media at low light conditions (no hood light). The different dilutions with different media are given in Table 4 below.

Cells were treated with the liposome dilutions (200 μL/well). For reference, a “no-cell control” (NOC) and a “no-treat control” were performed. The no-cell control is a well without cells treated with the highest liposome concentration, whereas the no-treat control (no-treatment) is a well containing cells treated only with growth media (CM or DMEM).

Incubation time was either 1, 3, 7 or 24 hours. At the end of the respective incubation time, the treatment medium was removed and the cells were washed with DPBS three times, the last DPBS wash was left in the well. The results of the fluorescence intensity measurements are shown in FIGS. 1-6.

2.2.2 Liposome Uptake into Growing Cell Cultures—Cell Imaging

C2C12 cells are seeded into an 8 chamber multi-well glass slide (Corning Life Sciences) at 5000-10000 cells/well (400-500 μL/well) in DMEM growth media supplemented with 10% (v/v) FBS. Cells are cultured until approximately 70% confluency with a homogeneous distribution is achieved (cell culture stage 1). A serial dilution of liposomes was prepared in the different media at low light conditions (no hood light). Cells are treated with liposome dilutions ranging from 1.5% to 50% (500 μL/well) and incubation time was 1 to 24 hours. At the end of the respective incubation time, the treatment medium is aspirated and each chamber well is washed for a total of three times using DPBS supplemented with 10% FBS (v/v) and the final DPBS-FBS wash is left in the well before the cells are imaged with a 20-60× objective and a LionHeart FX Automated Microscope (Agilent BioTek). Nile Red liposome signals in the cells are detected by imaging the cells under fluorescence detection mode at Excitation/Emission wavelengths of 586 nm and 647 nm, respectively. In parallel, the cells are also imaged in phase-contrast mode and the images are captured/overlayed/analysed using Gen5 Prime software (Agilent BioTek).

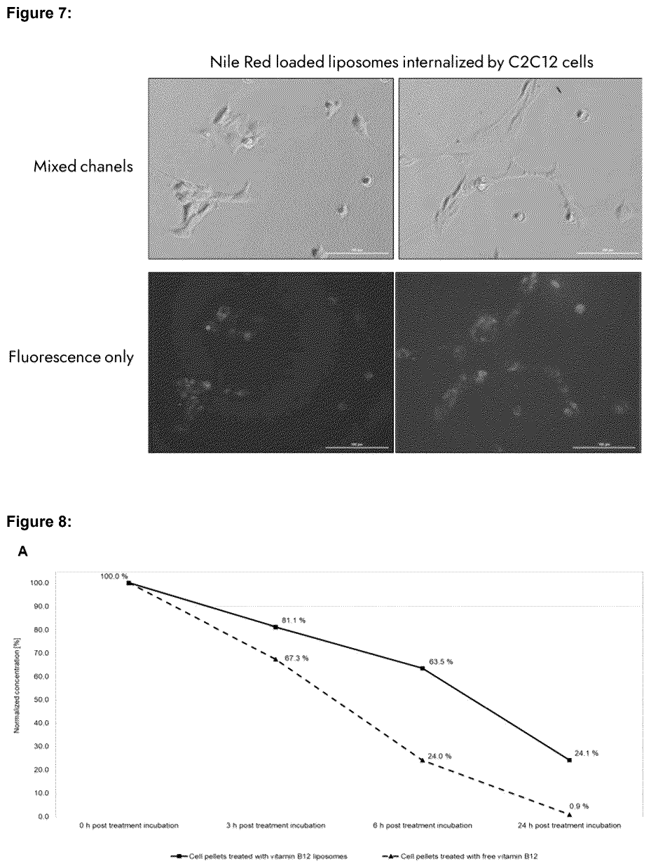

The results of the cell imaging are shown in FIG. 7.

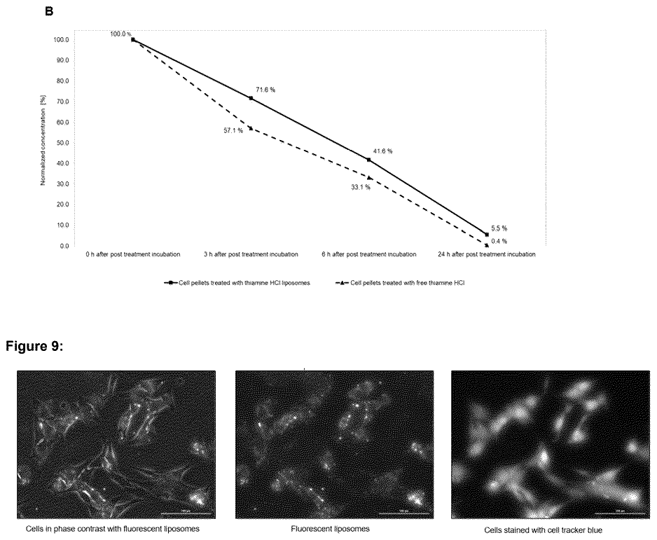

In another experiment, on day 0, mouse C2C12 cells were seeded in growth media (Dulbecco's Modified Eagle Medium (DMEM; Gibco; Cat #11995-065); supplemented with 10% Fetal Bovine Serum and 1× Penicillin-Streptomycin) at a density of 2000-5000 cells per chamber of an 8-chamber culture slide (BD-Falcon #354108) and grown at 37 °C for 48 hours. On Day 2, the master stock/suspension of fluorescent liposomes (e.g. nitrobenzenefurazan-labelled) is diluted by 6-fold into phenol-free DMEM containing 10% fetal bovine serum. The final liposome concentration of this diluted solution is 16.67%. Growth media is removed from the cells and replaced with 400 μl of liposome solution and the culture slide is returned to the cell incubator for 17-18 hours. On day 3, after the liposome incubation period, the liposome solution is removed from the cells and the cells are washed four times with 500-1000 μl serum-free, phenol-free DMEM. The liposome loaded cells are counterstained with a 500 μl solution containing 1× Cell Tracker Blue CMF₂HC (Invitrogen #C12881), which is prepared in serum-free, phenol-free DMEM (1:1000 dilution=1×) and incubated on the cells for 2-4 hours. After the incubation period the cells are washed four times with serum-free, phenol-free DMEM. The cells are then fixed by adding 500 μl of a PBS solution containing 4% paraformaldehyde (Thermo Scientific; Cat #J61899) for 20 minutes at room temperature. The slide is washed four times with 500-1000 μl PBS. After washing the chamber walls are removed from the culture slide using the chamber removal key. After chamber removal, several droplets of Epredia PermaFluor Mounting Medium are deposited on the slide and then the entire slide is covered with a coverslip. The mounting medium is allowed to cure in the dark at 4 degrees C for 24-48 hours. Day 4: after the mounting medium has cured, the cells are imaged via phase contrast and epifluorescence microscopy using 4×, 20×, and 40× objectives with a LionHeart FX imager (BioTek/Agilent). Signals from fluorescent liposomes are detected with a GFP filter set (Excitation 469 nm, Emission 525 nm) and cell staining with Cell Tracker Blue is detected using a DAPI filter set (Excitation 377 nm, Emission 447 nm). Cell Images were captured using Gen5 Microplate Reader and Imager software (BioTek/Agilent). The results of the cell imaging are shown in FIG. 9.

TABLE 4
Dilutions with different media

| | Growth | | Phospholipid |
|---|---|---|---|
| Sample | medium | Dilution | concentrations |
| No-cell control | CM | 1:2 | 1.700 mg/mL or |
| | | | 330 μg/well |
| No-cell control | DMEM | 1:2 | 1.700 mg/mL or |
| | | | 330 μg/well |
| No-treatment | CM | No treat | Cells without |
| control | | (medium | liposomes |
| | | only) | |
| No-treatment | DMEM | No treat | Cells without |
| control | | (medium | liposomes |
| | | only) | |
| Sample 1:2 | CM | 1:2 | 1.700 mg/mL or |
| | | | 330 μg/well |
| Sample 1:2 | DMEM | 1:2 | 1.700 mg/mL or |
| | | | 330 μg/well |
| Sample 1:4 | CM | 1:4 | 0.850 mg/mL or |
| | | | 165 μg/well |
| Sample 1:4 | DMEM | 1:4 | 0.850 mg/mL or |
| | | | 165 μg/well |
| Sample 1:8 | CM | 1:8 | 0.425 mg/mL or |
| | | | 82.5 μg/well |
| Sample 1:8 | DMEM | 1:8 | 0.425 mg/mL or |
| | | | 82.5 μg/well |
| Sample 1:16 | CM | 1:16 | 0.213 mg/mL or |
| | | | 41.3 μg/well |
| Sample 1:16 | DMEM | 1:16 | 0.213 mg/mL or |
| | | | 41.3 μg/well |
| Sample 1:32 | CM | 1:32 | 0.106 mg/mL or |
| | | | 20.6 μg/well |
| Sample 1:32 | DMEM | 1:32 | 0.106 mg/mL or |
| | | | 20.6 μg/well |
| Sample 1:64 | CM | 1:64 | 0.053 mg/mL or |
| | | | 10.3 μg/well |
| Sample 1:64 | DMEM | 1:64 | 0.053 mg/mL or |
| | | | 10.3 μg/well |
| Sample 1:128 | CM | 1:128 | 0.027 mg/mL or |
| | | | 5.2 μg/well |
| Sample1:128 | DMEM | 1:128 | 0.027 mg/mL or |
| | | | 5.2 μg/well |
| Sample 1:256 | CM | 1:256 | 0.013 mg/mL or |
| | | | 2.6 μg/well |
| Sample 1:256 | DMEM | 1:256 | 0.013 mg/mL or |
| | | | 2.6 μg/well |

Fluorescence of cells and of the treatment medium alone was detected in the plate reader using the following setting:
Fluorescence Endpoint, detection from bottom
Excitation: 554 nm, Emission: 638 nm; Gain 100 RFU (Relative Fluorescence Units)

### 2.2.3 Liposome Uptake and Retention/Metabolization of Exogenous Compounds in Cells

To test potential compound retention/metabolization effects, C2C12 cells were incubated for 17 h with either exogenous compound-loaded liposomes or with the free compound, applied at the same concentration, by methods described herein. Cells were washed with DPBS and incubated with complete medium for 3 h, 6 h, 24 h (post-incubation step) to promote normal cell metabolism. After the post-treatment incubation step, the treatment medium was removed and cells were washed twice with DPBS and harvested. Viability determination and cell counting of harvested cells were performed using a NucleoCounter NC-202 (ChemoMetec A/S, Allerod, Denmark). To determine the amount of exogenous compound taken up and/or associated with the cells, the cells were centrifuged at 130*g 5 min, washed with DPBS and snap-frozen. Samples of treatment media and cell pellets were analysed/quantified by ultra-performance liquid chromatography-mass spectrometry (UPLC-MS/MS). Samples were diluted with ethanol for vitamin B12 and with ethanol: water (50:50) for thiamine HCl. Cell pellets were dissolved using an ultrasonic bath.

The content of thiamine HCl or vitamin B12 was calculated using linear regression analysis of the calibration curve generated using a reference.

The comparison between the exogenous compound concentration found in cell pellets harvested immediately after treatment and those which had a post-treatment incubation step provides insights on the stability of compounds associated with cells, when applied in free form or when encapsulated. The results are shown in FIG. 8-A and B for two exogenous compounds vitamin B12 and thiamine HCl, respectively. The detected or analysed amount of the exogenous compound (encapsulated in liposome and free) was normalized to 100%. As shown in FIG. 8A, the rate of vitamin B12 clearance was higher (for all time points) for cells loaded with free vitamin B12 compared to vitamin B12 encapsulated in liposomes. Cells treated with vitamin B12 encapsulated in liposomes retained 81% and 64% of the 0 h concentration after 3 h and 6 h of post-treatment incubation step, respectively, while cell treated with free vitamin B12 only retained 67% and 24% at the same time points. This suggests that vitamin B12 encapsulated in liposomes is not only taken up and/or associated with cells but that cells can retain liposomes encapsulated vitamin B12 longer. A similar observation can also be made for the liposome encapsulated thiamine HCl compared to free thiamine HCl (FIG. 8B). Without being bound to any theory, this effect may be attributed to liposomes protecting the exogenous compounds from intracellular metabolization and thereby improving retention.

### 2.2.4 Viability of Cells Incorporating (Liposome Encapsulated) Exogenous Compounds

The data below shows cell count and viability determination for the different conditions within the vitamin B12 and thiamine HCl experiment. Cell counts of harvested cell pellets were compared for a given concentration and measured using the method described herein. Counts and viability of cells incubated with liposomes were comparable to untreated cells and both doubled in number overnight suggesting that vitamin B12- or thiamine-loaded liposomes do not decrease cellular viability and are not cytotoxic. The percentages are normalized to the control cells (100%).

### Vitamin B12

| | Normalized cell | Viability |
|---|---|---|
| | count [%] | [%] |
| 6 h incubation | | |
| Control cells (no treatment) | 100 | 100 |
| Cells treated with free vitamin B12 | 97 | 104 |
| Cells treated with liposomal vitamin B12 | 111 | 99 |
| 17 h incubation | | |
| Control cells | 100 | 100 |
| Cells treated with free vitamin B12 | 100 | 99 |
| Cells treated with liposomal vitamin B12 | 104 | 104 |

### Thiamine HCl

| | Normalized cell | Viability |
|---|---|---|
| | count (%) | [%] |
| 6 h incubation | | |
| Control cells (no treatment) | 100 | 100 |
| Cells treated with free thiamine HCl | 111 | 101 |
| Cells treated with liposomal thiamine HCl | 93 | 93 |
| 17 h incubation | | |
| Control cells | 100 | 100 |
| Cells treated with free thiamine HCl | 93 | 104 |
| Cells treated with liposomal thiamine HCl | 98 | 102 |

### Example 3—Use of Non-Human Animal Cells Derived from Oviparous Animals

Non-human animal cells are grown as described, for example, by Olivier, S. et al., (2010. EB66 cell line, a duck embryonic stem cell-derived substrate for the industrial production of therapeutic monoclonal antibodies with enhanced ADCC activity. MAbs, 2(4): 405-15), Madeline, B. et al., (2015. Culturing a duck ES-derived cell line in single-use bioreactors: A rapid, efficient, and cost-effective vaccine manufacturing system based on suspension culture. BioProcess International, 13), Silim, A., Azhary, M. A. S. Y. E., Roy, R. S., (1982. A Simple Technique for Preparation of Chicken-Embryo-Skin Cell Cultures. Avian Diseases, 26(1): 182-185); Chen, Y.-C. et al., (2019. In vitro culture and characterization of duck primordial germ cells. Poultry science, 98(4): 1820-1832.) Farzaneh, M., et al. (2017). The evolution of chicken stem cell culture methods. British Poultry Science, 58(6): 681-686).

Addition of supplement compositions and their harvest and analysis are done according to Example 2.

### Example 4—Use of Non-Human Animal Cells Derived from Stem Cells Harvested from Umbilical Cord, Bone Marrow, Placenta or Other Tissue

Non-human animal cells are grown as described by Pham, P. V. et al., (2014. Good manufacturing practice-compliant isolation and culture of human umbilical cord blood-derived mesenchymal stem cells. Journal of Translational Medicine, 12(1): 56) Smith, J. R. et al., (2016. Standardizing Umbilical Cord Mesenchymal Stromal Cells for Translation to Clinical Use: Selection of GMP-Compliant Medium and a Simplified Isolation Method. Stem Cells International, 2016:)

Addition of supplement compositions and their harvest and analysis are done according to Example 2.

### Example 5—Nanoparticles

### 5.1 Preparation of Loaded Nanoparticles

(Method M1) Lipid nanoparticles were prepared by weighing a wax (e.g. Palm oil) and an oil that is liquid at room temperature (e.g. Medium Chain Triglycerides, MCT) at a weight ratio between 90:10 and 50:50 wax:oil. Soy lecithin (90% PC) at a ratio of 98:2 wax+oil:soy Lecithin was added to the melt. The combined weight of the wax, oil and soy lecithin was around 10 gm. This mixture was heated above the melting temperature of the wax (about 70 °C). Up to 1 gm of exogenous compounds (e.g. 2,4-decadienal) can be mixed with the wax melt. This mixture formed the organic phase. Separately, a Polysorbate 80 solution containing 2.4 grams Polysorbate 80 in 87.4 gram distilled water was prepared and heated up to about 70 °C. This solution formed the aqueous phase. The organic phase was injected slowly into the aqueous phase under strong magnetic stirring (1200 rpm). After complete addition of the organic phase the formed emulsion was sonicated (Banson digital sonifier model 250) for 5 minutes and subsequently processed for about 10 minutes by high shear homogenization (Polytron 6100 D) at 12000 rpm. The resulting emulsion was cooled down to room temperature under ambient conditions in order to solidify the emulsion, forming the lipid nanoparticles. If required, unencapsulated flavors may be removed from the samples by using PD Miditrap G-25 gel filtration columns (Cytiva, Grens Switzerland). The samples are then subsequently filtered through 220 nm sterile filters.

(Method M2) An organic phase was prepared by dissolving wax (e.g. Palm oil) and an oil that is liquid at room temperature (e.g. medium chain triglycerides, MCT) at a weight ratio between 90:10 and 50:50 wax:oil in a mixture of chloroform:methanol (2:1). The total amount of lipids (wax+oil) was around 300 mg. Up to 100 mg of desired exogenous compounds (e.g. 2,4-decadienal) may be mixed with the dissolved wax and oil. The organic solvents were removed by rotary evaporation at about 65 °C at about 300 mbar vacuum. Residual organic solvent traces were then removed in a vacuum oven at about 40 °C and about 100 mbar for at least 15 hours. Next, a Polysorbate 80 solution was made by dissolving 300-600 mg of Polysorbate 80 in about 20 mL of distilled water and heating to about 65 °C. The heated solution was added to the dried organic phase and the mixture underwent 3 minutes high shear homogenization (Polytron 6100 D) at 12000 rpm. Then, the formed dispersion was sonicated by a probe sonicator (Banson digital sonifier model 250) for about 20 minutes at an amplitude setting of 50%. The resulting emulsion was cooled down to room temperature under ambient conditions in order to solidify the emulsion, forming the lipid nanoparticles. If required, unencapsulated flavors may be removed from the samples by using PD Miditrap G-25 gel filtration columns (Cytiva, Grens Switzerland). The samples are then subsequently filtered through 220 nm sterile filters.

### 5.2 Stability Data for Flavour-Loaded Nanoparticles

The stability of size or mean particle size (average diameter) and polydispersity index (PDI) were measured using the methods described before for the liposomes (e.g. dynamic light scattering). The results are listed in table 5. The flavour loaded-nanoparticles we analysed by GC-MS and the following amount of the loaded or encapsulated flavour (2,4-decadienal) was determined: method M1: 4490 μg/mL, method M2: 2426 μg/mL.

The GC-MS method to quantify loaded or encapsulated flavour was performed as follows: 2,4-decadienal was analyzed by gas chromatography-mass spectrometry selected ion monitoring (GC-MS SIM). For GC-MS SIM sample preparation, 200 μL of sample containing loaded delivery system (e.g. nanoparticles) was taken, internal GC-MS SIM standard in acetone was added and the volume was increased to 1 mL with acetone in order to extract all loaded flavors. The cell pellets may additionally be homogenized with a high shear mixer (Polytron PT 1200E, Kinematica, Eschbach Germany) for 1 minute. The samples were filtered through a 220 nm syringe filter.

The GC-MS SIM (GC=7890B, MS=5977B MSD, Agilent Technologies, Lautengartenstrasse 6, Basel) measurement sample injection volume was 1 μL. The content of loaded or encapsulated flavour was calculated using linear regression analysis of the calibration curve generated using a reference.

For the measurement of the mean particle size and PDI, 20-50 microliters of sample was diluted in the same dispersion medium up to a volume of 2 mL in a disposable cuvette. The cuvette was placed inside the Zetasizer Nano ZS90 (Malvern Panalytical) and measured by dynamic light scattering. Measurements were performed in triplicates and average values was considered.

TABLE 5
Average diameter measurements (Ø) and polydispersity index (PDI) of liposomes
with encapsulated exogenous compound, done by dynamic light scattering (DLS)

| Method of | | Day 0 | Day 2 | Day 3 | Day 4 | Day 7 |
|---|---|---|---|---|---|---|
| preparation (MX), | Exogenous | A. Ø | A. Ø | A. Ø | A. Ø | A. Ø |
| conditions | compound | B. PDI | B. PDI | B. PDI | B. PDI | B. PDI |
| M1 | 2,4- | A. 135.8 | A. 200.8 | A. 200.0 | A. 200.4 | A. 188.4 |
| Organic phase: | decadienal | B. 0.107 | B. 0.269 | B. 0.265 | B. 0.251 | B. 0.250 |
| 8 g palm oil | | | | | | |
| 2 g MCT | | | | | | |
| 0.2 g Soy lecithin | | | | | | |
| 1 g 2,4- | | | | | | |
| Decadienal | | | | | | |
| Aqueous phase: | | | | | | |
| 2.4 g | | | | | | |
| Polysorbate 80 | | | | | | |
| 87.4 g dH₂O | | | | | | |
| M2 | 2,4- | A. 126.6 | N/M | N/M | N/M | N/M |
| Organic phase: | decadienal, | B. 0.255 | | | | |
| 210 mg palm oil | | | | | | |
| 90 g MCT | | | | | | |
| 10 mg 2,4-DD | | | | | | |
| Aqueous phase: | | | | | | |
| 300 mg | | | | | | |
| Polysorbate 80 | | | | | | |
| 20 mL dH2O | | | | | | |

N/M = not measured

1. A method for producing a non-human animal-derived cell composition, comprising:
contacting a single type or a plurality of non-human animal-derived cells with at least one exogenous supplement selected from flavour materials, flavour precursors, flavour enhancers, colours, colour precursors, bioactive compounds or other food-related additives and mixtures thereof.

2. The method according to claim 1, wherein the exogenous supplement is comprised in a carrier.

3. The method according to claim 2, wherein the carrier is a nanocarrier, a nanoparticle, a micelle, a liposome or a vesicle.

4. The method according to claim 2, wherein the carrier is a nanoparticle or a liposome.

5. The method according to claim 2, wherein the carrier is a liposome.

6. (canceled)

7. (canceled)

8. (canceled)

9. (canceled)

10. The method according to claim 2, wherein the carrier has a mean particle size less than 500 nm or is a liposome with a size polydispersity of below 0.3.

11. (canceled)

12. The method according to claim 1, wherein the cells are contacted with the exogenous supplement comprised in a carrier at times of either low cell differentiation rate or high cell differentiation rate.

13. (canceled)

14. The method according to claim 1, wherein the cells are contacted with the exogenous supplement comprised in a carrier at times of either low cell division rate or high cell division rate.

15. (canceled)

16. (canceled)

17. (canceled)

18. (canceled)

19. (canceled)

20. (canceled)

21. (canceled)

22. (canceled)

23. The method according to claim 1, wherein the time of contact is sufficient such that at least 20% of the cells contain an exogenous supplement comprised in a carrier at a site selected from the cell intracellular space, intramembrane space, on the membrane and any combination thereof.

24. (canceled)

25. (canceled)

26. (canceled)

27. A non-human animal-derived cell composition comprising a plurality of non-human animal-derived cells, wherein at least 20% of the cells comprise at least one exogenous supplement wherein the cell composition is characterized by having improved organoleptic properties and/or having a meat-like flavour, and wherein the exogenous supplement is comprised in a carrier, wherein the at least one exogenous supplement is selected from the group consisting of flavours materials, flavours, flavour precursors, flavour enhancers, colours, colour precursors, bioactive compounds, or other food-related additives and mixtures thereof.

28. (canceled)

29. The non-human animal-derived cell composition according to claim 27, wherein the carrier is a nanocarrier, a nanoparticle, a micelle, a liposome or a vesicle.

30. The non-human animal-derived cell composition according to claim 27, wherein the carrier is a nanoparticle or a liposome.

31. (canceled)

32. (canceled)

33. (canceled)

34. (canceled)

35. (canceled)

36. (canceled)

37. (canceled)

38. (canceled)

39. The non-human animal-derived cell composition according to claim 27, wherein the at least one exogenous supplement comprised in a carrier is present in the cells at a site selected from the group consisting of the cell intracellular space, intramembrane space, on the membrane and any combination thereof.

40. (canceled)

41. (canceled)

42. The non-human animal-derived cell composition according to claim 27, wherein the plurality of cells contains an amount of the at least one exogenous supplement of about 0.01 mg/100 g cells to about 100 mg/100 g cells.

43. The non-human animal-derived cell composition of claim 27, wherein the at least one exogenous supplement composition is contained in an amount of about 0.05 mg/100 g cells to about 500 mg/100 g cells.

44. (canceled)

45. An enriched cell culture medium comprising at least one exogenous supplement, wherein the supplement is in an amount sufficient to confer an organoleptic effect to cells cultivated in the medium, according to the method of claim 1, and wherein the exogenous supplement is comprised in a carrier and wherein the at least one exogenous supplement is selected from the group consisting of flavour materials, flavour precursors, flavour enhancers, colours, colour precursors, bioactive compounds, or other food-related additives and any combination thereof.

46. (canceled)

47. An enriched scaffold comprising at least one exogenous supplement, wherein the supplement is in an amount sufficient to confer an organoleptic effect to cells cultivated in the presence of the scaffold, according to the method of claim 1, and wherein the exogenous supplement is comprised in a carrier and wherein the at least one exogenous supplement selected from the group consisting of flavour materials, flavours, flavour precursors, flavour enhancers, colours, colour precursors, bioactive compounds, or other food-related additives and any combination thereof.

48. (canceled)

49. The enriched cell culture medium according to claim 45, wherein the carrier is a nanocarrier, a nanoparticle, a micelle, a liposome or a vesicle.

50. A method of utilizing the cell composition according to claim 27, wherein the method comprises adding the cell composition to flavour food or beverage comestibles.

51. The method according to claim 50, wherein the comestible comprises cultivated meat, meat analogues or meat products.

52. (canceled)
